SEQUENCE LISTING (6 sequences)

SEQ ID NO: 1 - protein, 333 aa, Homo sapiens [sequence omitted]

SEQ ID NO: 2 - protein, 175 aa, Homo sapiens
APRSVDWREKGYVTPVKNQGQCGSCWAFSATGALEGQMFRKTGRLISLSEQNLVDCSGPQGNEGCNGGLMDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFEST

SEQ ID NO: 3 - protein, 42 aa, Homo sapiens
NNKYWLVKNSWGEEWGMGGYVKMAKDRRNHCGIASAASYPTV

SEQ ID NO: 4 - protein, 10 aa, Homo sapiens
LYGMNEEGWR

SEQ ID NO: 5 - protein, 11 aa, Homo sapiens
ALDFAVGEYNK

SEQ ID NO: 6 - protein, 10 aa, Homo sapiens
ESDTSYVSLK

(19) United States
(12) Patent Application Publication
(10) Pub. No.: US 2014/0302509 A1
(43) Pub. Date: Oct. 9, 2014

(54) PROCATHEPSIN L AND CATHEPSIN L AS BIOMARKERS FOR ISCHEMIA

(71) Applicant: Pronota N.V., Zwijnaarde (BE)

(72) Inventors: Piet Moerman, Deurle (BE); Griet Vanpoucke, Ingooigem (BE); Gregoire Thomas, Lokeren (BE); Koen Kas, Schilde (BE)

(21) Appl. No.: 14/351,477

(22) PCT Filed: Oct. 12, 2012
(86) PCT No.: PCT/EP2012/070320

§ 371 Date: Apr. 11, 2014

Related U.S. Application Data
(60) Provisional application No. 61/547,191, filed on Oct. 14, 2011.

(30) Foreign Application Priority Data
Oct. 14, 2011 (EP) 11185145.7

(51) Int. Cl. G01N 33/53; C12Q 1/68

(52) U.S. Cl. CPC G01N 33/5308; C12Q 1/686
USPC 435/6.12; 435/7.1; 436/501

(57) ABSTRACT

The present invention relates to procathepsin L, cathepsin L or a fragment thereof as a biomarker for ischemia. The present invention further relates to methods for diagnosing, predicting, prognosticating and/or monitoring ischemia based on measuring said biomarker, and to related kits, devices and uses thereof.

### FIELD OF THE INVENTION

The invention relates to protein- and/or peptide-based biomarkers useful for diagnosing, predicting, prognosticating and/or monitoring diseases and conditions, in particular ischemia in subjects; and to related methods, kits and devices.

### BACKGROUND OF THE INVENTION

In many diseases and conditions, a favourable outcome of prophylactic and/or therapeutic treatments is strongly correlated with early and/or accurate prediction, diagnosis, prognosis and/or monitoring of a disease or condition. Therefore, there exists a continuous need for additional and preferably improved means for early and/or accurate prediction, diagnosis, prognosis and/or monitoring of diseases and conditions to guide the treatment choices.

Critically ill patients presenting in intensive care units (ICU) or emergency departments (ED) are mainly patients following major surgery or trauma. It is known that the physiological response of the critically ill patient to a stress or disease process will largely determine his outcome including survival or death. Therefore, it is of major importance to monitor the physiological state of the patient. Monitoring the health state of a patient might help in the early diagnosis of a change in a physiological parameter and can provide guidance to the medical practitioner towards appropriate therapy.

However, there are many physiological variables which can be assessed and these can further vary in complexity as well as degrees of invasiveness. Of major importance in critically ill patients is the maintenance of normal aerobic metabolism and thus the maintenance of viable cell function. However, measuring the degree of tissue oxygenation is notoriously difficult. It is possible to measure the oxygen content in the venous blood draining individual tissues, which can be compared with those of the arterial blood. In practice however this approach can only be applied to a limited number of organs where the relevant blood samples can be taken such as the lung, the liver and the brain. There are also microelectrode systems available which can measure PO₂ and pH as well as some electrolytes including potassium which is released from ischemic tissues but again in practice these can only be placed in relatively few sites for instance in muscle. Laser Doppler flow monitoring can further be used to assess local blood flow but this does not look at oxygen uptake by the tissues.

As an alternative approach, lactate has been used as a marker for tissue hypoxia in critically ill patients. A relationship between lactate concentration and cumulative oxygen debt has been established (Weil and Afifi, 1970, Circulation, 41, 989-1001). Furthermore, the use of lactate to assess the efficacy of therapy has been suggested (Rivers et al, 2001, N. Eng. J. Med., 2001, 345, 1368-77). However, there are several drawbacks for lactate as a clinical marker of tissue hypoxia. Sample collection, the stability of the samples, the metabolic effects of blood cells and technical problems may affect the interpretation of lactate concentrations. Furthermore, other processes not related to tissue hypoxia and subsequent anaerobic metabolism such as liver insufficiency can result in increased blood lactate levels complicating clinical interpretation and therapy in cases of raised lactate levels. In addition, in some situations plasma lactate does not increase despite its local formation due to exclusion of the area from perfusion or lactate levels do not correspond to energetic failure due to intoxications.

Dependable and preferably early detection and intervention is critical to effective treatment of critically ill patients. Consequently, provision of further, alternative and preferably improved markers and tools for diagnosis, prediction, prognosis and/or monitoring the health state of critically ill patients continues to be of prime importance.

### SUMMARY OF THE INVENTION

Having conducted extensive experiments and tests, the inventors have found that levels of the protein procathepsin L, cathepsin L or a fragment thereof in blood samples are closely indicative of ischemia. As illustrated in the example section, procathepsin L levels were studied 24 hours after surgery in clinical samples from 100 cardiac surgery patients, who underwent coronary artery bypass graft (CABG) or heart valve repair or heart valve replacement surgery. Cardiac surgery induces system-wide hypoxia which can lead to organ ischemia. The most sensitive organ to this is the kidney, leading to (acute) kidney injury. Procathepsin L levels following surgery were elevated in patients who developed significant AKI or died within 90 days after surgery. Most patients died of heart failure or cardiogenic shock, i.e. insufficient perfusion. Accordingly, the inventors have realised procathepsin L, cathepsin L or a fragment thereof as a new biomarker advantageous for evaluating the degree of ischemia in subjects.

Ischemia is an absolute or relative shortage of the blood supply to an organ, i.e. a shortage of oxygen, glucose and other blood-borne fuels. A relative shortage means the mismatch of blood supply (oxygen/fuel delivery) and blood request for adequate metabolism of tissue. Ischemia results in tissue damage because of a lack of oxygen and nutrients and, ultimately, this can cause severe damage because of the potential for a build-up of metabolic wastes.

Ischemia can also be described as an inadequate flow of blood to a part of the body, caused by constriction or blockage of the blood vessels supplying it. Since oxygen is mainly bound to hemoglobin in red blood cells, insufficient blood supply causes tissue to become hypoxic, or, if no oxygen is supplied at all, anoxic. In very aerobic tissues such as heart and brain, at body temperature necrosis due to ischemia usually takes about 3-4 minutes before becoming irreversible. Complete cessation of oxygenation of such organs for more than 20 minutes typically results in irreversible damage.

Ischemia is a feature of heart diseases, transient ischemic attacks, cerebrovascular accidents, ruptured sensitive to inadequate blood supply. Ischemia in brain tissue, for example due to stroke or head injury, causes a process called the ischemic cascade to be unleashed, in which proteolytic enzymes, reactive oxygen species, and other harmful chemicals damage and may ultimately kill brain tissue.

In view of the potentially drastic effects that a lack of oxygen may have on an organ (and ultimately, the patient), there is an advantage to being able to detect ischemia (as inadequate blood flow) at a stage where the likelihood of hypoxia and associated tissue damage and complications may be prevented or substantially reduced.

Thus, the present enables the detection of the degree, level or progress of ischemia due to the fact that Cathepsin-L levels appear to show an almost linear correlation with the degree or severity of the ischemic event in the subject. This is in contrast with commonly used Lactate levels, since these levels show a large grey zone between no ischemia and severe ischemia, making it virtually unable to specifically grade the severity of an ischemic event.

In a first aspect, the present invention therefore relates to the use, in particular in vitro use, of procathepsin L, cathepsin L or a fragment thereof as a blood biomarker for diagnosing, predicting, prognosticating and/or monitoring the degree of ischemia in a subject. Such use is advantageous because it allows inter alia early detection of ischemia and hence provides early guidance to the medical practitioner about the installation of a therapeutic intervention in a critically ill subject or a subject at risk of developing ischemia-related complications and will allow the medical practitioner to assess the impact of such therapy. Furthermore, such use advantageously helps optimizing the training program of a subject under revalidation or of a sportsperson.

Using procathepsin L, cathepsin L or a fragment thereof as a marker for ischemia may be particularly useful in subjects diagnosed with ischemia, or known or expected to be at risk of developing ischemia. The subjects which might benefit from the use of procathepsin L, cathepsin L or a fragment thereof as a biomarker include critically ill patients such as without any limitation patients presenting in intensive care units (ICU) or emergency departments (ED) with serious trauma, sepsis, systemic inflammatory response syndrome (SIRS) or chronic obstructive pulmonary disease (COPD) with or without an acute exacerbation, or patients having undergone surgery and more particularly cardiac surgery, in whom the incidence of ischemia is highly probable. Furthermore, such subjects include subjects at risk of developing ischemia-related complications, i.e. subjects having one or more of the conditions selected from atherosclerosis, diabetes, obesity, ischemic heart disease, chronic heart failure, heart valve problems, lipid disorders, lipoprotein disorders, and claudicatio. Using procathepsin L, cathepsin L or a fragment thereof as a biomarker may further be useful in subjects under revalidation or in sportspersons, when trying to ameliorate, improve or optimize their health status.

In an embodiment, the present invention provides the use of procathepsin L, cathepsin L or a fragment thereof as a blood biomarker for diagnosing, predicting, prognosticating and/or monitoring ischemia in a subject, wherein said diagnosis, prediction, prognosis and/or monitoring ischemia comprises assessing the degree of ischemia in a subject. Preferably, the degree of ischemia in a subject is assessed in accordance with lactate levels. More preferably, the degree of ischemia is assessed as being: (i) no ischemia, ii) low degree of ischemia with reversible or reparable physiological outcome, or (iii) high degree of ischemia with potential irreversible or irreparable physiological damage, morbidity or mortality.

Generally, lactate concentrations measured in blood which are above 4-5 mmol/l are predictive of ischemia-related complications of a subject, while in healthy patients values for lactate are around 1±0.5 mmol/l (Valenza et al., Crit. Care, 9(6), 588-593). Lactate concentrations above 2 mmol/l but below 5 mmol/l do not allow predicting the outcome of the patient, nor are they used for therapeutic decision making. Thus, the range of lactate levels between 2 and 5 mmol/l, also referred to as the “grey zone” of lactate concentrations, leave the practitioner in doubt as whether to start any specific therapeutic intervention.

Advantageously, the present invention enables the distinction of subjects with no ischemia or low levels of ischemia with reversible or reparable physiological outcome from subjects with high levels of ischemia with irreversible or irreparable physiological damage, morbidity or mortality. As illustrated in the example section, procathepsin L levels advantageously allowed satisfactory discrimination between subjects with a favourable outcome and subjects with ischemia-related complications even in the grey zone of lactate concentrations, i.e. when the lactate concentration ranged between 2 and 5 mmol/l.

In an embodiment, the present invention provides the use of procathepsin L, cathepsin L or a fragment thereof as a blood biomarker for diagnosing, predicting, prognosticating and/or monitoring ischemia in a subject in combination with lactate as a biomarker. Such use advantageously improves the predictive power of lactate as a biomarker.

In a second aspect, the present invention provides a method, in particular an in vitro method, for diagnosing, predicting, prognosticating and/or monitoring ischemia in a subject, wherein the examination phase of the method comprises measuring the quantity and/or activity of procathepsin L, cathepsin L or a fragment thereof in a blood sample from the subject. The subject can be, for instance, a critically ill subject, a subject at risk of developing ischemia-related complications, a subject under revalidation, or a sportsperson.

For example but without limitation, an elevated quantity and/or activity (i.e., a deviation) of procathepsin L, cathepsin L or a fragment thereof in a sample from a subject compared to a reference value representing the diagnosis or prediction of no ischemia or low levels of ischemia with reversible or reparable physiological outcome or representing a good prognosis for ischemia indicates that the subject has or is at risk of having high levels of ischemia with irreversible or irreparable physiological damage or indicates a poor prognosis for ischemia in the subject such as morbidity or mortality.

Hence, methods applying the principles of the present invention advantageously allow the diagnosis or prediction of ischemia in a subject, thereby inter alia providing guidance to the medical practitioner in order to select the appropriate therapeutic intervention, or in order to adapt the therapeutic intervention for instance in a critically ill patient.

Furthermore, the methods applying the principles of the present invention allow the medical practitioner to monitor the disease progress by measuring the level of procathepsin L, cathepsin L or a fragment thereof in a sample of the patient. For example, a decrease in the quantity and/or activity (i.e., a deviation) of procathepsin L, cathepsin L or a fragment thereof compared to a prior quantity and/or activity of procathepsin L, cathepsin L or a fragment thereof (e.g., at the time of the admission to ED or ICU) indicates the ischemia in the subject is improving or has improved, while an increase in the quantity and/or activity (i.e., a deviation) of procathepsin L, cathepsin L or a fragment thereof as compared to a prior quantity and/or activity (i.e., a deviation) of procathepsin L, cathepsin L or a fragment thereof (e.g., at the time of the admission to ED or ICU) indicates the ischemia in the subject has worsened or is worsening. Such worsening could result in severe ischemia-related complications of the patient, such as those selected from the group consisting of: acute kidney injury (AKI), cardiogenic shock, myocardial infarction, heart failure, death, amputation or removal of the damaged area, organ or limb, brain infarction and its neurological deficits, and any organ damage or failure.

Accordingly, the invention further provides a method, in particular an in vitro method, for diagnosing, predicting, prognosticating and/or monitoring acute kidney injury in a subject, wherein the examination phase of the method comprises measuring the quantity of procathepsin L, cathepsin L or a fragment thereof in a blood sample from the subject.

The invention also provides a method, in particular an in vitro method, for predicting mortality in critical ill patients, wherein the examination phase of the method comprises measuring the quantity of procathepsin L, cathepsin L or a fragment thereof in a blood sample from the patient.

Throughout the present disclosure, methods suitable for monitoring any one condition or disease as taught herein can inter alia allow the prediction of the occurrence of the condition or disease, or monitoring the progression, aggravation, alleviation or recurrence of the condition or disease, or response to treatment or to other external or internal factors, situations or stressors, etc. Advantageously, monitoring methods as taught herein may be applied in the course of a medical treatment of the subject, preferably medical treatment aimed at alleviating the so-monitored condition or disease. Such monitoring may be comprised, e.g., in decision making whether a patient may be discharged, needs a change in therapeutic intervention or needs further hospitalisation.

Similarly, throughout the present disclosure, methods suitable for the prognosis of any one condition or disease as taught herein can inter alia allow the prognosis of the occurrence of the condition or disease, or to prognosticate the progression, aggravation, alleviation or recurrence of the condition or disease, or response to treatment or to other external or internal factors, situations or stressors, etc.

Further provided according to the invention are the present methods as taught herein, wherein said diagnosis, prediction, prognosis and/or monitoring ischemia comprises assessing the degree of ischemia in the subject. Preferably, the degree of ischemia in a subject is assessed in accordance with lactate levels. This implies that lactate levels can be used as an additional reference for assessing the degree of ischemia in the subject (cf. e.g. FIGS. 2 and 4, where lactate and pro-cathepsin-L levels are measured in subjects with different degrees of ischemia). More preferably, the degree of ischemia is assessed as being: (i) no ischemia, ii) low degree of ischemia with reversible or reparable physiological outcome, or (iii) high degree of ischemia with potential irreversible or irreparable physiological damage, morbidity or mortality. Such methods have the advantage in that they help the medical practitioner to select the appropriate therapeutic intervention (cf. FIG. 2, indicating an almost linear correlation between Cathepsin-L levels and the three different groups of ischemia severity). Such methods also provide guidance to the practitioner in steering the therapeutic intervention for instance in determining the dose or regimen of the therapy to be applied to the subject.

As mentioned above, the present invention provides a method for diagnosing, predicting, prognosticating and/or monitoring ischemia in a subject, wherein the examination phase of the method comprises measuring the quantity and/or activity of procathepsin L, cathepsin L or a fragment thereof in a blood sample from the subject. In an embodiment, the examination phase of the method further comprises measuring the quantity of lactate in the blood sample from the subject. Such methods advantageously improve the interpretation of lactate levels in a sample from the subject.

As illustrated in the example section, the inventors have found that procathepsin L levels in critically ill subjects 24 hours post-surgery were significantly higher in those subjects with ischemia-related complications, including acute kidney injury and death, 90 days after surgery compared to those subjects with a favourable outcome 90 days after surgery. Consequently, the inventors have realised procathepsin L, cathepsin L or a fragment thereof as a biomarker that is advantageous for distinguishing subjects with a likely favourable outcome from subjects with ischemia-related complications.

Accordingly, further provided according to the present invention are the uses or methods as taught herein, wherein said diagnosis, prediction, prognosis and/or monitoring ischemia comprises distinguishing subjects with a likely favourable outcome from subjects with ischemia-related complications. Such uses and methods advantageously help the medical practitioner to determine how the subject such as a critically ill patient or a patient under revalidation, copes with the ischemic stress. For example and without limitation, such uses and methods allow the medical practitioner to monitor a subject under revalidation and to distinguish no ischemia or low levels of ischemia due to increased exercise from high levels of ischemia with irreversible or irreparable physiological damage, morbidity or mortality.

The uses or methods as defined herein can hence be used for determining and/or steering the therapeutic intervention in the subject, for assessing the impact of the therapeutic intervention; or for measuring the success of ischemic preconditioning in a subject who will undergo surgery or transplantation.

As illustrated in the experimental section, ischemia-related complications in critically ill subjects are associated with elevated levels of procathepsin L, cathepsin L or a fragment thereof. Consequently, diagnosis or prediction of ischemia-related complications can in particular be associated with an elevated level of procathepsin L, cathepsin L or a fragment thereof.

For example but without limitation, an elevated quantity and/or activity (i.e., a deviation) of procathepsin L, cathepsin L or a fragment thereof in a sample from a subject compared to a reference value representing the diagnosis, prediction or prognosis of a likely favourable outcome indicates that the subject has a comparably greater risk of ischemia-related complications.

As further shown in the examples, the inventors have found that procathepsin L levels in critically ill subjects were significantly higher in those subjects who will have died within 90 days post-surgery compared to those subjects who will have remained alive at 90 days post-surgery. Consequently, the inventors have realised procathepsin L, cathepsin L or a fragment thereof as a biomarker that is advantageous for predicting or prognosticating mortality in critically ill patients.

Hence, also provided are the uses or methods as taught herein, wherein said diagnosis, prediction, prognosis and/or monitoring of ischemia comprises predicting or prognosticating mortality in a subject.

Such uses and methods for predicting or prognosticating mortality may be preferably performed for any critically ill subject or for instance once the critically ill subject is diagnosed or predicted with ischemia, more preferably upon the initial (first) diagnosis or prediction of ischemia.

As shown in the experimental section, increased mortality rate in critically ill subjects is associated with elevated levels of procathepsin L, cathepsin L or a fragment thereof. Consequently, prediction or prognostication of increased mortality (increased risk or chance of death within a predetermined time interval) can in particular be associated with an elevated level of procathepsin L, cathepsin L or a fragment thereof.

For example but without limitation, an elevated quantity and/or activity (i.e., a deviation) of procathepsin L, cathepsin L or a fragment thereof in a sample from a subject compared to a reference value representing the prediction prognosis of a given mortality (i.e., a given, such as a normal, risk or chance of death within a predetermined time interval) indicates that the subject has a comparably greater risk of deceasing within said time interval.

Without limitation, mortality may be suitably expressed as the chance of a subject to decease within an interval of for example several hours, days, weeks or months from the time of performing a prediction or prognostication method, e.g., within about 3 hours, 6 hours, 12 hours, 24 hours, or within about 2 days, 3 days, 4 days, 5 days, 6 days or within about 1 week, 2 weeks, 3 weeks or within about 1 month or within about 2 months, 3 months, 4 months, 5 months, or 6 months from the time of performing the prediction or prognostication method.

It shall be appreciated that the finding of increased chance of death in a subject can guide therapeutic decisions, for instance the therapeutic intervention to treat the critically ill subject.

Atherosclerosis, diabetes, obesity, ischemic heart disease, chronic heart failure, heart valve problems, lipid disorders, lipoprotein disorders, and claudicatio represent major risk factors for developing ischemia. Hence, the present uses or methods may be preferably employed in such patients and patient populations, i.e., in subjects having or being at risk of having one or more of the conditions selected from atherosclerosis, diabetes, obesity, ischemic heart disease, chronic heart failure, heart valve problems, lipid disorders, lipoprotein disorders, and claudicatio (such as, e.g., in a screening setup).

Hence, further provided according to the present invention are the uses or methods as taught herein, wherein said diagnosis, prediction, prognosis and/or monitoring ischemia comprises assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio.

Also provided are the methods as taught herein, wherein said diagnosis, prediction, prognosis and/or monitoring ischemia comprises screening or monitoring a subject of developing ischemia-related complications, based on one or more of the following risk factors: atherosclerosis, diabetes, obesity, ischemic heart disease, chronic heart failure, heart valve problems, lipid disorders, lipoprotein disorders, and claudicatio, wherein said method is used at regular time points to follow the level of procathepsin L, cathepsin L or a fragment thereof during the life of the subject.

Further intended herein according to the present invention are the uses or methods as defined herein for determining the therapeutic intervention in the subject. The recitation “determining the therapeutic intervention” refers to deciding which therapeutic intervention would be appropriate for the subject. The therapeutic intervention can be any intervention which stabilizes or improves the inadequate perfusion or oxygenation causing the ischemia such as for instance one or more of increased oxygen administration, mechanical ventilation, mechanical circulatory support, pharmaceutical circulatory support, fluid administration, dialysis assistance, administration of inotropic agents, red blood cell transfusion, and administration of paralytic agents, sedatives or analgesics.

For instance and without limitation, the uses or methods as defined herein may be useful in determining the therapeutic intervention taking into account other physiological parameters. Such physiological parameters may include temperature, arterial blood pressure, central venous pressure, stroke volume, oxygen delivery index (DO2I), blood gas pressures pO2 and pCO2, lactate levels, electrolytes and bicarbonate levels, blood pH, cardiac output, tidal volume, and heart frequency.

Also provided according to the present invention are the uses or methods as described herein for steering the therapeutic intervention in the subject. The recitation “steering the therapeutic intervention” may refer without limitation to one or more of deciding to start or stop the therapeutic intervention in the subject, deciding to change or adapt the therapeutic intervention of the subject to new conditions or health state of the subject, deciding whether the subject would (still) benefit from a therapeutic intervention or not, or deciding the dose or regimen of the therapeutic intervention.

Accordingly, also disclosed are the methods as taught herein for determining whether a subject is or is not (such as, for example, still is, or is no longer) in need of a therapeutic intervention or therapy to treat inadequate perfusion or oxygenation causing the ischemia, comprising: (i) measuring the quantity and/or activity of procathepsin L, cathepsin L or a fragment thereof in the sample from the subject; (ii) comparing the quantity and/or activity of procathepsin L, cathepsin L or a fragment thereof measured in (i) with a reference value of the quantity and/or activity of procathepsin L, cathepsin L or a fragment thereof, said reference value representing a known prediction, diagnosis and/or prognosis of ischemia or no ischemia; (iii) finding a deviation or no deviation of the quantity and/or activity of procathepsin L, cathepsin L or a fragment thereof measured in (i) from said reference value; (iv) inferring from said finding the presence or absence of a need for a therapy to treat ischemia. In an embodiment, said method may further comprise measuring the quantity of lactate in the blood sample from the subject. A therapy may be particularly indicated where steps (i) to (iii) allow for a conclusion that the subject has or is at risk of having ischemia or has a poor prognosis for ischemia, such as for example but without limitation, where the quantity and/or activity of procathepsin L in the sample from the subject is elevated (i.e., a deviation) compared to a reference value representing the prediction or diagnosis of no ischemia. Without limitation, a patient having ischemia upon admission to or during a stay in a medical care centre may be tested as taught herein for the necessity of continuing a treatment of said ischemia, and may be discharged when such treatment is no longer needed or is needed only to a given limited extent.

Hence, also envisaged are the uses or methods as defined herein for use in indicating the success of the therapeutic intervention in the subject. Further envisaged are the uses or methods as defined herein for use in assisting to decide about discharging the subject from the hospital.

Any one use or method as taught herein may preferably allow for sensitivity and/or specificity (preferably, sensitivity and specificity) of at least 50%, at least 60%, at least 70% or at least 80%, e.g., ≧85% or ≧90% or ≧95%, e.g., between about 80% and 100% or between about 85% and 95%.

Reference throughout this specification to “diseases and/or conditions” encompasses any such diseases and conditions as disclosed herein insofar consistent with the context of such a recitation, in particular ischemia.

The uses or methods as taught herein for predicting, diagnosing, prognosticating and/or monitoring ischemia may be used in individuals who have not yet been diagnosed as having such (for example, preventative screening), or who have been diagnosed as having such, or who are suspected of having such (for example, display one or more characteristic symptoms), or who are at risk of developing such (for example, genetic predisposition; presence of one or more developmental, environmental or behavioural risk factors). The present methods may also be used to detect various stages of progression or severity of the diseases or conditions. The present methods as taught herein may also be used to detect response of the diseases or conditions to prophylactic or therapeutic treatments or other interventions such as one or more of diet and life style changes and weight loss. The present methods can furthermore be used to help the medical practitioner in deciding upon worsening, status-quo, partial recovery, or complete recovery of the patient from the diseases or conditions, resulting in either further treatment or observation or in discharge of the patient from medical care centre.

Any one of the herein described uses or methods may be employed for population screening (such as, e.g., screening in a general population or in a population stratified based on one or more criteria, e.g., age, gender, ancestry, occupation, presence or absence of risk factors of ischemia, etc.).

Also intended herein according to the present invention are the uses or method as taught herein, wherein said monitoring ischemia comprises optimizing the training program of a subject. The subject can be a sportsperson or a subject under revalidation such as a subject at risk of developing ischemia-related complications. Such uses or methods advantageously enable the monitoring of and improvement in the health status or physical condition of the subject.

Further disclosed are the methods as defined herein, wherein said monitoring ischemia comprises screening or monitoring the health status in a subject such as in a subject under revalidation or in a sportsperson, wherein said method is used at regular time points to follow the level of procathepsin L, cathepsin L or a fragment thereof during a certain period, such as during the revalidation period or during the training period of the subject.

The methods applying the principles of the present invention allow the medical practitioner or the sport coach to monitor the progress of the health status of the subject by measuring the level of procathepsin L, cathepsin L or a fragment thereof in a sample of the subject. For example, a low level of procathepsin L, cathepsin L or a fragment thereof indicates no ischemia or a healthy degree of ischemia beneficial to the subject, while a high level of procathepsin L, cathepsin L or a fragment thereof indicates high levels of ischemia with irreversible or irreparable physiological damage, morbidity or mortality. Such high levels of procathepsin L, cathepsin L or a fragment thereof indicate that the revalidation or training should be reduced or stopped.

As mentioned above, the present invention provides a method for diagnosing, predicting, prognosticating and/or monitoring ischemia in a subject, wherein the examination phase of the method comprises measuring the quantity and/or activity of procathepsin L, cathepsin L or a fragment thereof in a blood sample from the subject. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

As used throughout this specification, measuring the levels of procathepsin L, cathepsin L or a fragment thereof and/or other biomarker(s) such as lactate in a sample from a subject may particularly denote that the examination phase of a method comprises measuring the quantity and/or activity of procathepsin L, cathepsin L or a fragment thereof and/or other biomarker(s) in the sample from the subject. One understands that methods of prediction, diagnosis, prognosis and/or monitoring of diseases and conditions generally comprise an examination phase in which data is collected from and/or about the subject.

In an embodiment, a method for predicting, diagnosing and/or prognosticating ischemia comprises the steps of: (i) measuring the quantity and/or activity of procathepsin L, cathepsin L or a fragment thereof in a sample from the subject; (ii) comparing the quantity and/or activity of procathepsin L, cathepsin L or a fragment thereof measured in (i) with a reference value of the quantity and/or activity of procathepsin L, cathepsin L or a fragment thereof, said reference value representing a known prediction, diagnosis and/or prognosis of ischemia or no ischemia; (iii) finding a deviation or no deviation of the quantity and/or activity of procathepsin L, cathepsin L or a fragment thereof measured in (i) from the reference value; and (iv) attributing said finding of deviation or no deviation to a particular prediction, diagnosis and/or prognosis of ischemia or no ischemia in the subject. Preferably, said diagnosis, prediction, prognosis and/or monitoring ischemia comprises assessing the degree of ischemia in the subject; distinguishing subjects with favourable outcome from subjects with ischemia-related complications; assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio; or optimizing the training program of a subject. In said cases, said reference value may be linked to a particular degree of ischemia in respective reference patient populations.

The method for predicting, diagnosing and/or prognosticating ischemia, and in particular such method comprising steps (i) to (iv) as set forth in the previous paragraph, may be performed for a subject at two or more successive time points and the respective outcomes at said successive time points may be compared, whereby the presence or absence of a change between the prediction, diagnosis and/or prognosis of ischemia at said successive time points is determined. The method thus allows the monitoring of a change in the prediction, diagnosis and/or prognosis of ischemia in a subject over time.

In an embodiment, a method for monitoring ischemia comprises the steps of: (i) measuring the quantity and/or activity of procathepsin L, cathepsin L or a fragment thereof in samples from a subject from two or more successive time points; (ii) comparing the quantity and/or activity of procathepsin L, cathepsin L or a fragment thereof between the samples as measured in (i); (iii) finding a deviation or no deviation of the quantity and/or activity of procathepsin L, cathepsin L or a fragment thereof between the samples as compared in (ii); and (iv) attributing said finding of deviation or no deviation to a change in ischemia in the subject between the two or more successive time points. Preferably, said monitoring ischemia comprises assessing the degree of ischemia in the subject, distinguishing subjects with favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

The uses or methods as taught herein thus allow the monitoring of ischemia in a subject over time for example to monitor the degree of ischemia in a subject, the monitoring of a change in the outcome of a subject over time, or the monitoring of a change in the health status of the subject over time. Furthermore, the uses or methods as taught herein also allow the monitoring of a change in the risk of developing ischemia-related complications over time in a subject having one or more of the conditions selected from atherosclerosis, diabetes, obesity, ischemic heart disease, chronic heart failure, heart valve problems, lipid disorders, lipoprotein disorders, and claudicatio.

In the present methods, the measurement of procathepsin L, cathepsin L or a fragment thereof may also be combined with the assessment of one or more further biomarkers or clinical parameters relevant for ischemia.

By means of example and not limitation, biomarkers useful in the present methods may include lactate; inflammatory markers such as C-reactive protein, interleukine-6 (IL-6), interleukine-8 (IL-8), growth differentiation factor 15 (GDF-15); kidney function markers such as cystatin C and neutrophil gelatinase associated lipocalin (NGAL); cardiac function markers such as natriuretic peptides, for example, pro-brain natriuretic peptide (proBNP), N-terminal fragment of pro-brain natriuretic peptide (NT-proBNP), brain natriuretic peptide (BNP), atrial natriuretic peptide (ANP), pro-atrial natriuretic peptide (pro-ANP); cardiac ischemia markers such as cardiac troponin T and cardiac troponin I. Biomarkers useful in the present methods may further include fragments or precursors of any one of the aforementioned biomarkers. Preferably, a biomarker useful in the present methods as taught herein is lactate.

Hence, further provided is the present method for predicting, diagnosing and/or prognosticating ischemia, wherein the examination phase of the method comprises measuring the quantity lactate in the blood sample from the subject. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject. Such methods advantageously improve the interpretation of the results of the lactate measurements in the sample of the subject. Such methods inter alia help the medical practitioner to determine and/or steer the therapeutic intervention.

Also disclosed is a method for predicting, diagnosing and/or prognosticating ischemia in a subject comprising the steps: (i) measuring the quantity and/or activity of procathepsin L, cathepsin L or a fragment thereof and the quantity of lactate in the sample from the subject; (ii) using the measurements of (i) to establish a subject profile of the quantity and/or activity of procathepsin L, cathepsin L or a fragment thereof and the quantity of lactate; (iii) comparing said subject profile of (ii) to a reference profile of the quantity and/or activity of procathepsin L, cathepsin L or a fragment thereof and the quantity of lactate, said reference profile representing a known prediction, diagnosis and/or prognosis of ischemia or no ischemia; (iv) finding a deviation or no deviation of the subject profile of (ii) from the reference profile; (v) attributing said finding of deviation or no deviation to a particular prediction, diagnosis and/or prognosis of ischemia in the subject. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Applying said method for predicting, diagnosing and/or prognosticating ischemia in a subject at two or more successive time points allows for the monitoring of ischemia. Preferably, said monitoring ischemia comprises assessing the degree, stage or level of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Also provided according to the present invention are the uses or methods as taught herein for improving the interpretation of lactate levels in a sample from the subject. As mentioned above, lactate concentrations above 2 mmol/l but below 5 mmol/I do not allow the prediction of the outcome of the patient and thus leave the practitioner in doubt as whether to start any therapeutic intervention. In an exemplary but non-limiting experiment, procathepsin L levels allowed satisfactory discrimination between favourable and ischemia-related complications when the lactate concentration ranged between 2 and 5 mmol/l.

The present methods may employ reference values for the quantity and/or activity of procathepsin L, cathepsin L or a fragment thereof, which may be established according to known procedures previously employed for other biomarkers. Such reference values may be established either within (i.e., constituting a step of) or external to (i.e., not constituting a step of) the methods of the present invention as defined herein.

Accordingly, in an embodiment, the present method for diagnosing, predicting, prognosticating and/or monitoring ischemia in a subject may comprise a step of establishing a reference value for the quantity and/or activity of procathepsin L, said reference value may represent either (a) a prediction or diagnosis of the absence of ischemia or a good prognosis thereof, or (b) a prediction or diagnosis of ischemia or a poor prognosis thereof. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Hence, further provided is a method for establishing a reference value for the quantity and/or activity of procathepsin L, cathepsin L or a fragment thereof, said reference value representing:

(a) a prediction or diagnosis of the absence of the diseases or conditions as taught herein or a good prognosis thereof, or
(b) a prediction or diagnosis of the diseases or conditions as taught herein or a poor prognosis thereof,
comprising:
(i) measuring the quantity and/or activity of procathepsin L, cathepsin L or a fragment thereof in:
(i a) one or more samples from one or more subjects not having the respective diseases or conditions or not being at risk of having such or having a good prognosis for such, or
(i b) one or more samples from one or more subjects having the respective diseases or conditions or being at risk of having such or having a poor prognosis for such, and
   (ii) storing the quantity and/or activity of procathepsin L, cathepsin L or a fragment thereof
(ii a) as measured in (i a) as the reference value representing the prediction or diagnosis of the absence of the respective diseases or conditions or representing the good prognosis therefore, or
(ii b) as measured in (i b) as the reference value representing the prediction or diagnosis of the respective diseases or conditions or representing the poor prognosis therefore.

The present methods may otherwise employ reference profiles for the quantity and/or activity of procathepsin L, cathepsin L or a fragment and the presence or absence and/or quantity of one or more other biomarkers such as lactate, which may be established according to known procedures previously employed for other biomarkers. Such reference profiles may be established either within (i.e., constituting a step of) or external to (i.e., not constituting a step of) the present methods as taught herein.

Accordingly, the present method for diagnosing, predicting, prognosticating and/or monitoring ischemia in a subject may comprise a step of establishing a reference profile for the quantity and/or activity of procathepsin L, cathepsin L or a fragment and the presence or absence and/or quantity of said one or more other biomarkers such as lactate, said reference profile representing either (a) a prediction or diagnosis of the absence of the diseases or conditions as taught herein or a good prognosis therefore, or (b) a prediction or diagnosis of the diseases or conditions as taught herein or a poor prognosis therefore. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Further disclosed is a method for establishing a reference profile for the quantity and/or activity of procathepsin L, cathepsin L or a fragment thereof and the presence or absence and/or quantity of one or more other biomarkers such as lactate useful for the methods of the present invention as taught herein, said reference profile representing:

(a) a prediction or diagnosis of the absence of the respective diseases or conditions or a good prognosis therefore, or
(b) a prediction or diagnosis of the respective diseases or conditions or a poor prognosis therefore,
comprising:
(i) measuring the quantity and/or activity of procathepsin L, cathepsin L or a fragment thereof and the presence or absence and/or quantity of said one or more other biomarkers such as lactate in:
(i a) one or more samples from one or more subjects not having the respective diseases or conditions or not being at risk of having such or having a good prognosis for such; or
(i b) one or more samples from one or more subjects having the respective diseases or conditions or being at risk of having such or having a poor prognosis for such;
   (ii)
(ii a) using the measurements of (i a) to create a profile of the quantity and/or activity of procathepsin L, cathepsin L or a fragment thereof and the presence or absence and/or quantity of said one or more other biomarkers; or
(ii b) using the measurements of (i b) to create a profile of the quantity and/or activity of procathepsin L, cathepsin L or a fragment thereof and the presence or absence and/or quantity of said one or more other biomarkers;
   (iii)
(iii a) storing the profile of (ii a) as the reference profile representing the prediction or diagnosis of the absence of the respective diseases or conditions or representing the good prognosis therefore; or
(iii b) storing the profile of (ii b) as the reference profile representing the prediction or diagnosis of the respective diseases conditions or representing the poor prognosis therefore.

Further provided is a method for establishing a base-line of procathepsin L, cathepsin L or a fragment thereof or a reference value of procathepsin L, cathepsin L or a fragment thereof in a subject, comprising: (i) measuring the quantity and/or activity of procathepsin L, cathepsin L or a fragment thereof in the sample from the subject at different time points wherein the subject is not suffering from the diseases or conditions as taught herein, and (ii) calculating the range or mean value of the subject, which is the base-line of procathepsin L, cathepsin L or a fragment thereof or the reference value of procathepsin L, cathepsin L or a fragment thereof for said subject.

Also provided is the use or method according to any one of the embodiments described herein, for predicting ischemia-related conditions and outcomes in pregnant women such as: placental insufficiency, placental thrombosis, placental infarction, abruption placentae, Intra Uterine Growth Retardation, Small for Gestational Age children, neurological or intellectual sequellae in the babie(s), spontaneous abortion, premature contractions, premature labor and delivery, fetal deformations, fetal infection, mors in utero, or low birth weight.

Further provided is the use or method according to any one of embodiments described herein in combination with lactate for detecting liver cell insufficiency by differentiating between abnormal lactate production and abnormal lactate metabolism, wherein abnormal lactate production is indicated when both levels of lactate and procathepsin L, cathepsin L or a fragment thereof are elevated, and wherein abnormal lactate metabolism is indicated when lactate levels are elevated but the level of procathepsin L, cathepsin L or a fragment thereof is in the normal range.

Preferably, the subject as intended in any one of the present methods may be human.

In the present methods, the quantity of procathepsin L, cathepsin L or a fragment thereof can be measured using: a binding agent capable of specifically binding to procathepsin L, cathepsin L or a fragment thereof respectively, an immunoassay technology, a mass spectrometry analysis method, a chromatography method, using RNA analysis tools such as northern blotting, or (quantitative) RT-PCR, or a combination of said methods.

The quantity of procathepsin L, cathepsin L or a fragment thereof and/or the presence or absence and/or quantity of the one or more other biomarkers such as lactate may be measured by any suitable technique such as may be known in the art. For example, the quantity of procathepsin L, cathepsin L or a fragment thereof and/or the presence or absence and/or quantity of the one or more other biomarkers such as lactate may be measured using, respectively, a binding agent capable of specifically binding to procathepsin L, cathepsin L or a fragment thereof and/or to fragments thereof, and a binding agent capable of specifically binding to said one or more other biomarkers such as lactate. For example, the binding agent may be an antibody, aptamer, spiegelmer (L-aptamer), photoaptamer, protein, peptide, peptidomimetic or a small molecule. For example, the quantity of procathepsin L, cathepsin L or a fragment thereof and/or the presence or absence and/or quantity of the one or more other biomarkers may be measured using an immunoassay technology or a mass spectrometry analysis method or a chromatography method, or a combination of said methods.

The activity of procathepsin L, cathepsin L or a fragment thereof can be measured using any suitable technique such as may be known in the art. The activity of procathepsin L, cathepsin L or a fragment thereof can be measured using for instance but without limitation an assay that utilizes the preferred cathepsin L substrate sequence phenylalanine(Phe)-arginine(Arg) labeled with a fluorescent probe such as amino-4-trifluoromethyl coumarin (Abcam, Cambridge, UK) or cresyl violet (AbD Serotec, Dusseldorf, Germany).

Also provided according to the present invention is a kit for diagnosing, predicting, prognosticating and/or monitoring ischemia in a subject, the kit comprising (i) means for measuring the quantity and/or activity of procathepsin L, cathepsin L or a fragment in a sample from the subject. Optionally and preferably, the kit further comprises (ii) a reference value of the quantity and/or activity of procathepsin L, cathepsin L or a fragment or means for establishing said reference value, wherein said reference value represents a known diagnosis, prediction and/or prognosis of ischemia or no ischemia. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject. The kit thus allows one to: measure the quantity and/or activity of procathepsin L, cathepsin L or a fragment in the sample from the subject by means (i); compare the quantity and/or activity of procathepsin L, cathepsin L or a fragment measured by means (i) with the reference value of (ii) or established by means (ii); find a deviation or no deviation of the quantity and/or activity of procathepsin L, cathepsin L or a fragment measured by means (i) from the reference value of (ii); and consequently attribute said finding of deviation or no deviation to a particular prediction, diagnosis and/or prognosis of ischemia or no ischemia in the subject.

The kits for performing the present methods as described herein may additionally comprise means for measuring the quantity of lactate or any other suitable biomarker in a sample of the subject.

Hence, a further embodiment provides a kit for diagnosing, predicting, prognosticating and/or monitoring ischemia in a subject, the kit comprising (i) means for measuring the quantity and/or activity of procathepsin L in a sample from the subject and (ii) means for measuring the quantity of lactate in a sample from the subject, and optionally and preferably (iii) means for establishing a subject profile of the quantity and/or activity of procathepsin L and the quantity of lactate, and optionally and preferably (iv) a reference profile of the quantity and/or activity of procathepsin L and the quantity of lactate, or means for establishing said reference profile, said reference profile representing a known diagnosis, prediction and/or prognosis of ischemia or no ischemia. Such kit thus allows one to: measure the quantity and/or activity of procathepsin L and the quantity of lactate in a sample from the subject by respectively means (i) and (ii); establish (e.g., using means included in the kit or using suitable external means) a subject profile of the quantity and/or activity of procathepsin L and the quantity of lactate based on said measurements; compare the subject profile with the reference profile of (iv) or established by means (iv); find a deviation or no deviation of said subject profile from said reference profile; and consequently attribute said finding of deviation or no deviation to a particular prediction, diagnosis and/or prognosis of ischemia or no ischemia in the subject. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject. Such kits thus allow one to: measure the quantity and/or activity of procathepsin L, cathepsin L or a fragment thereof and the quantity of lactate in a sample from the subject by respectively means (i) and (ii); establish (e.g., using means included in the kit or using suitable external means) a subject profile of the quantity and/or activity of procathepsin L, cathepsin L or a fragment thereof and the quantity of lactate based on said measurements; compare the subject profile with the reference profile of (iv) or established by means (iv); find a deviation or no deviation of said subject profile from said reference profile; and consequently attribute said finding of deviation or no deviation to a particular diagnosis, prediction and/or prognosis of ischemia or no ischemia.

The means for measuring the quantity of procathepsin L, cathepsin L or a fragment thereof and/or the quantity of lactate in the present kits may comprise, respectively, one or more binding agents capable of specifically binding to procathepsin L, cathepsin L or a fragment thereof, and/or one or more binding agents capable of specifically binding to lactate. For example, any one of said one or more binding agents may be an antibody, aptamer, spiegelmer (L-aptamer), photoaptamer, protein, peptide, peptidomimetic or a small molecule. For example, any one of said one or more binding agents may be advantageously immobilised on a solid phase or support. The means for measuring the quantity of procathepsin L, cathepsin L or a fragment thereof and/or the quantity of lactate in the present kits may employ an immunoassay technology, or mass spectrometry analysis technology, or chromatography technology, or a combination of said technologies.

Disclosed is thus also a kit for diagnosing, predicting, prognosticating and/or monitoring ischemia in a subject as described herein comprising: (a) one or more binding agents capable of specifically binding to procathepsin L, cathepsin L or a fragment thereof; (b) preferably, a known quantity or concentration of procathepsin L, cathepsin L or a fragment thereof (e.g., for use as controls, standards and/or calibrators); (c) preferably, a reference value of the quantity of procathepsin L, cathepsin L or a fragment thereof, or means for establishing said reference value. Said components under (a) and/or (c) may be suitably labelled as taught elsewhere in this specification. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Also disclosed is a kit for diagnosing, predicting, prognosticating and/or monitoring ischemia in a subject as taught herein comprising: (a) one or more binding agents capable of specifically binding to procathepsin L, cathepsin L or a fragment thereof; (b) one or more binding agents capable of specifically binding to lactate; (c) preferably, a known quantity or concentration of procathepsin L, cathepsin L or a fragment thereof and a known quantity or concentration of lactate (e.g., for use as controls, standards and/or calibrators); (d) preferably, a reference profile of the quantity of procathepsin L, cathepsin L or a fragment thereof and the quantity of lactate, or means for establishing said reference profiles. Said components under (a), (b) and/or (c) may be suitably labelled as taught elsewhere in this specification. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Further disclosed is the use of the kit as described herein for diagnosing, predicting, prognosticating and/or monitoring ischemia in a subject as taught herein. Preferably, said diagnosis, prediction, prognosis and/or monitoring ischemia comprises assessing the degree of ischemia in the subject, distinguishing subjects with a likely favourable outcome from subjects with ischemia-related complications, assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio, or optimizing the training program of a subject.

Also disclosed are reagents and tools useful for measuring procathepsin L, cathepsin L or a fragment thereof and optionally the one or more other biomarkers concerned herein, particularly lactate.

Hence, disclosed is a protein, polypeptide or peptide array or microarray comprising (a) procathepsin L, cathepsin L and/or a fragment thereof, preferably a known quantity or concentration of said procathepsin L, cathepsin L and/or fragment thereof; and (b) optionally and preferably, one or more other biomarkers such as lactate, preferably a known quantity or concentration of said one or more other biomarkers such as lactate.

Also disclosed is a binding agent array or microarray comprising: (a) one or more binding agents capable of specifically binding to procathepsin L, cathepsin L or a fragment thereof, preferably a known quantity or concentration of said binding agents; and (b) optionally and preferably, one or more binding agents capable of specifically binding to one or more other biomarkers such as lactate, preferably a known quantity or concentration of said binding agents.

Also disclosed are kits as taught herein configured as portable devices, such as, for example, bed-side devices, for use at home or in clinical settings.

A related aspect thus provides a portable testing device capable of measuring the quantity and/or activity of procathepsin L, cathepsin L or a fragment thereof in a sample from a subject comprising: (i) means for obtaining a sample from the subject, (ii) means for measuring the quantity and/or activity of procathepsin L, cathepsin L or a fragment thereof in said sample, and (iii) means for visualising the quantity and/or activity of procathepsin L, cathepsin L or a fragment thereof measured in the sample.

In an embodiment, the means of parts (ii) and (iii) may be the same, thus providing a portable testing device capable of measuring the quantity and/or activity of procathepsin L, cathepsin L or a fragment thereof in a sample from a subject comprising (i) means for obtaining a sample from the subject; and (ii) means for measuring the quantity and/or activity of procathepsin L, cathepsin L or a fragment thereof in said sample and visualising the quantity and/or activity of procathepsin L, cathepsin L or a fragment thereof measured in the sample.

In an embodiment, said visualising means is capable of indicating whether the quantity and/or activity of procathepsin L, cathepsin L or a fragment thereof in the sample is above or below a certain threshold level and/or whether the quantity and/or activity of procathepsin L, cathepsin L or a fragment thereof in the sample deviates or not from a reference value of the quantity and/or activity of procathepsin L, cathepsin L or a fragment thereof, said reference value representing a known diagnosis, prediction and/or prognosis of ischemia or no ischemia. Hence, the portable testing device may suitably also comprise said reference value or means for establishing the reference value.

In an embodiment, the threshold level is chosen such that the quantity and/or activity of procathepsin L, cathepsin L or a fragment thereof in the sample above said threshold level indicates that the subject has or is at risk of having ischemia or indicates a poor prognosis for such in the subject, and the quantity and/or activity of procathepsin L, cathepsin L or a fragment thereof in the sample below said threshold level indicates that the subject does not have or is not at risk of having ischemia or indicates a good prognosis for such in the subject.

In an embodiment, the portable testing device comprises a reference value representing the prediction or diagnosis of the absence of ischemia or representing a good prognosis for such, or comprises means for establishing said reference value, and an elevated quantity and/or activity of procathepsin L, cathepsin L or a fragment thereof in the sample from the subject compared to said reference value indicates that the subject has or is at risk of having ischemia or indicates a poor prognosis for such in the subject. In another embodiment, the portable testing device comprises a reference value representing the prediction or diagnosis of ischemia or representing a poor prognosis for such, or comprises means for establishing said reference value, and a comparable quantity and/or activity of procathepsin L, cathepsin L or a fragment thereof in the sample from the subject compared to said reference value indicates that the subject has or is at risk of having ischemia or indicates a poor prognosis for such in the subject.

In a further embodiment, the measuring (and optionally visualisation) means of the portable testing device may comprise a solid support having a proximal and distal end, comprising:—a sample application zone in the vicinity of the proximal end;—a reaction zone distal to the sample application zone; and—a detection zone distal to the reaction zone;—optionally control standards comprising protein or peptide fragments of procathepsin L, cathepsin L or a fragment thereof, whereby said support has a capillary property that directs a flow of fluid sample applied in the application zone in a direction from the proximal end to the distal end; and—optionally comprising a fluid source improving the capillary flow of a more viscous sample.

The reaction zone may comprise one or more bands of specific binding molecules for procathepsin L, cathepsin L or a fragment thereof, conjugated to a detection agent, which specific binding molecule conjugate for procathepsin L, cathepsin L or a fragment thereof is disposed on the solid support such that it can migrate with the capillary flow of fluid; and wherein the detection zone comprises one or more capture bands comprising a population of specific molecules, which population of specific molecules for procathepsin L, cathepsin L or a fragment thereof is immobilised on the solid support.

The reaction zone may additionally comprise one or more bands of capture specific binding molecules for procathepsin L, cathepsin L or a fragment thereof in an amount sufficient to prevent a threshold quantity and/or activity of specific binding molecule conjugates of procathepsin L, cathepsin L or a fragment thereof to migrate to the detection zone. Alternatively, said device additionally comprises means for comparing the amount of captured specific binding molecule conjugate of procathepsin L, cathepsin L or a fragment thereof with a threshold value.

Other aspects relate to the realisation that procathepsin L, cathepsin L or a fragment thereof may be a valuable target for therapeutic and/or prophylactic interventions in ischemia.

Hence, also disclosed herein are any one and all of the following:

(1) an agent that is able to modulate the level and/or the activity of procathepsin L, cathepsin L or a fragment thereof for use as a medicament, preferably for use in the treatment of any one disease or condition as taught herein;
(2) use of an agent that is able to modulate the level and/or the activity of procathepsin L, cathepsin L or a fragment thereof for the manufacture of a medicament for the treatment of any one disease or condition as taught herein; or use of an agent that is able to modulate the level and/or the activity of procathepsin L, cathepsin L or a fragment thereof for the treatment of any one disease or condition as taught herein;
(3) a method for treating any one disease or condition as taught herein in a subject in need of such treatment, comprising administering to said subject a therapeutically or prophylactically effective amount of an agent that is able to modulate the level and/or the activity of procathepsin L, cathepsin L or a fragment thereof;
(4) The subject matter as set forth in any one of (1) to (3) above, wherein the agent is able to reduce or increase the level and/or the activity of procathepsin L, cathepsin L or a fragment thereof, preferably to reduce the level and/or the activity of procathepsin L, cathepsin L or a fragment thereof.
(5) The subject matter as set forth in any one of (1) to (4) above, wherein said agent is able to specifically bind to procathepsin L, cathepsin L or a fragment thereof.

(6) The subject matter as set forth in any one of (1) to (5) above, wherein said agent is an antibody or a fragment or derivative thereof; a polypeptide; a peptide; a peptidomimetic; an aptamer, spiegelmer (L-aptamer); a photoaptamer; or a chemical substance, preferably an organic molecule, more preferably a small organic molecule. Preferably, the agent is a chemical substance, for instance, a cathepsin L inhibitor such as N-(1-Naphthalenylsulfonyl)-Ile-Trp-aldehyde, Z-Phe-Tyr(tBu)-diazomethylketone, or Z-Phe-Tyr-aldehyde.

(7) The subject matter as set forth in any one of (1) to (4) above, wherein the agent is able to reduce or inhibit the expression of procathepsin L, cathepsin L or a fragment thereof, preferably wherein said agent is an antisense agent; a ribozyme; or an agent capable of causing RNA interference.
(8) The subject matter as set forth in any one of (1) to (4) above, wherein said agent is able to reduce or inhibit the level and/or activity of procathepsin L, cathepsin L or a fragment thereof, preferably wherein said agent is a recombinant or isolated deletion construct of the polypeptide of procathepsin L, cathepsin L or a fragment thereof having a dominant negative activity over the native procathepsin L, cathepsin L or a fragment thereof.
(9) An assay to select, from a group of test agents, a candidate agent potentially useful in the treatment of any one disease or condition as taught herein, said assay comprising determining whether a tested agent can modulate, such as increase or reduce and preferably reduce, the level and/or activity of procathepsin L, cathepsin L or a fragment thereof.
(10) The assay as set forth in (9) above, further comprising use of the selected candidate agent for the preparation of a composition for administration to and monitoring of the prophylactic and/or therapeutic effect thereof in a non-human animal model, preferably a non-human mammal model, of any one disease or condition as taught herein.
(11) The agent isolated by the assay as set forth in (10) above.
(12) A pharmaceutical composition or formulation comprising a prophylactically and/or therapeutically effective amount of one or more agents as set forth in any one of (1) to (8) or (10) above, or a pharmaceutically acceptable N-oxide form, addition salt, prodrug or solvate thereof, and further comprising one or more of pharmaceutically acceptable carriers.
(13) A method for producing the pharmaceutical composition or formulation as set forth in (12) above, comprising admixing said one or more agents with said one or more pharmaceutically acceptable carriers.

Said condition or disease as set forth in any one of (1) to (13) above is ischemia.

Also contemplated is thus a method (a screening assay) for selecting an agent capable of specifically binding to procathepsin L, cathepsin L or a fragment thereof (e.g., gene or protein) comprising: (a) providing one or more, preferably a plurality of test binding agents for procathepsin L, cathepsin L or a fragment thereof; (b) selecting from the test binding agents for procathepsin L, cathepsin L or a fragment thereof of (a) those which bind to procathepsin L, cathepsin L or a fragment thereof; and (c) counter-selecting (i.e., removing) from the test binding agents for procathepsin L, cathepsin L or a fragment thereof selected in (b) those which bind to any one or more other, unintended or undesired, targets.

Alternatively, one could envisage an inhibitor of the molecules responsible for the processing of procathepsin L, cathepsin L or a fragment thereof.

Binding between test binding agents for procathepsin L, cathepsin L or a fragment thereof and procathepsin L, cathepsin L or a fragment thereof may be advantageously tested by contacting (i.e., combining, exposing or incubating) said procathepsin L, cathepsin L or fragment thereof with the test binding agents for procathepsin L, cathepsin L or a fragment thereof under conditions generally conducive for such binding. For example and without limitation, binding between test binding agents for procathepsin L, cathepsin L or a fragment thereof and said procathepsin L, cathepsin L or fragment thereof may be suitably tested in vitro; or may be tested in host cells or host organisms comprising said procathepsin L, cathepsin L or fragment thereof and exposed to or configured to express the test binding agents for procathepsin L, cathepsin L or a fragment thereof.

Without limitation, the binding agents for procathepsin L, cathepsin L or a fragment thereof or the modulating agents for procathepsin L, cathepsin L or a fragment thereof may be capable of binding procathepsin L, cathepsin L or a fragment thereof or modulating the activity and/or level of the procathepsin L, cathepsin L or a fragment thereof in vitro, in a cell, in an organ and/or in an organism.

In the screening assays as set forth in any one of (9) and (10) above, modulation of the activity and/or level of the procathepsin L, cathepsin L or a fragment thereof by test modulating agents for procathepsin L, cathepsin L or a fragment thereof may be advantageously tested by contacting (i.e., combining, exposing or incubating) said procathepsin L, cathepsin L or fragment thereof (e.g., gene or protein) with the test modulating agents for procathepsin L, cathepsin L or a fragment thereof under conditions generally conducive for such modulation. By means of example and not limitation, where modulation of the activity and/or level of the procathepsin L, cathepsin L or a fragment thereof results from binding of the test modulating agents for procathepsin L, cathepsin L or a fragment thereof to the procathepsin L, cathepsin L or fragment thereof, said conditions may be generally conducive for such binding. For example and without limitation, modulation of the activity and/or level of the procathepsin L, cathepsin L or a fragment thereof by test modulating agents for procathepsin L, cathepsin L or a fragment thereof may be suitably tested in vitro; or may be tested in host cells or host organisms and exposed to or configured to express the test modulating agents for procathepsin L, cathepsin L or a fragment thereof.

As well contemplated are:
procathepsin L, cathepsin L or a fragment thereof for use as a medicament, preferably for use in the treatment of any one disease or condition as taught herein;
use of procathepsin L, cathepsin L or a fragment thereof for the manufacture of a medicament for the treatment of any one disease or condition as taught herein;
use of procathepsin L, cathepsin L or a fragment thereof for the treatment of any one disease or condition as taught herein;
a method for treating any one disease or condition as taught herein in a subject in need of such treatment, comprising administering to said subject a therapeutically or prophylactically effective amount of procathepsin L, cathepsin L or a fragment thereof;
   particularly wherein said condition or disease may be ischemia.

These and further aspects and preferred embodiments are described in the following sections and in the appended claims.

### BRIEF DESCRIPTION OF THE DRAWINGS

FIG. 1 illustrates sequences of full length procathepsin L (SEQ ID NO.1) and the cathepsin L heavy chain (SEQ ID NO. 2), and light chain (SEQ ID NO.3) fragments. The peptide detected by the MASSterclass™ technology is bold underlined (SEQ ID NO.4). The signal peptide is indicated as the N-terminal bold sequence in the depicted full length procathepsin L sequence. The peptides depicted as SEQ ID NO. 5 and 6 represent respectively the peptides detected by the MASSterclass™ technology from the Cystatin-C and C-reactive protein (CRP) reference biomarkers used.

FIG. 2 illustrates the stepwise increase in procathepsin L levels in relation to lactate levels as measure of ischemia. Lactate levels are binned in <2 mmol/L (normal; no ischemia); 2-5 mmol/L: elevated lactate levels and >5 mmol/L (severely elevated levels indicative of significant ischemia)

FIG. 3 illustrates the increases in procathepsin L levels observed after surgery. Levels as measured by MASSterclass before and after surgery are shown and lines indicate the levels in the same patient. Post-surgery levels were normalized to pre-surgery levels. Black lines illustrate patients with ischemia-related complications (death or AKI), grey line illustrate patients with favourable outcome. The cut-off fold increase for maximum accuracy of procathepsin L to predict ischemia-related complications is indicated.

FIG. 4 shows a graph illustrating the predictive power of the combination of procathepsin L levels and lactate levels in predicting ischemia-related complications 24 hours after surgery in 100 patients. Ischemia-related complications is indicated by a star (*) for patients that died during follow up and a hollow circle (O) for patients who developed AKI, filled (black) circles represent patients with a favourable outcome.

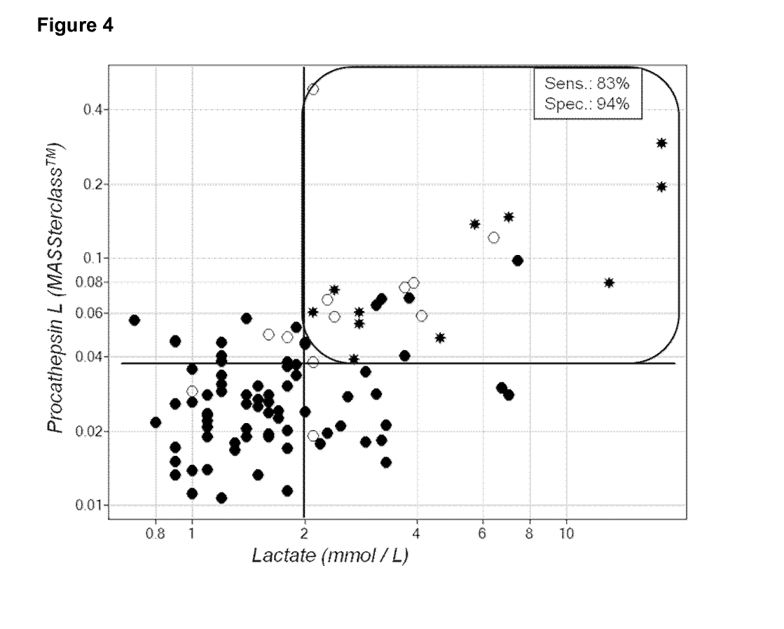

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The inventors show that procathepsin L, cathepsin L or a fragment thereof is a valuable biomarker for ischemia in a subject. The term “biomarker” is widespread in the art and may broadly denote a biological molecule and/or a detectable portion thereof whose qualitative, quantitative, or qualitative and quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition.

Reference herein to “disease(s) and/or condition(s) as taught herein” or a similar reference encompasses any such diseases and conditions as disclosed herein insofar consistent with the context of such a recitation, and in particular ischemia.

The terms “ischemia” or “ischaemia” or “ischemic stress” can be used interchangeably herein and generally refer to a disease or condition characterized by a restriction in blood supply, i.e. a shortage of oxygen, glucose and other blood-borne nutrients, with resultant damage or dysfunction of tissue. The ischemia can be one or more of renal ischemia, myocardial ischemia, brain ischemia, mesenteric ischemia, ischemic colitis, ischemic stroke, limb ischemia and cutaneous ischemia. The ischemia can be chronic or acute.

Signs and symptoms of ischemia may include without limitation any one or more of claudicatio; cold feet or fingers with or without pricking or numbing; angina pectoris with or without radiating signs such as numbness in left arm or hand, back pain or neck sensitations (increase in excitability of neurons, leading to pain); leg cramps; anuria; signs of stroke such as headache, numbness in limbs, paralysis or involuntary muscular contractions; and eye ischemia such as sudden blindness, retinopathy, intraocular bleeding or aneurisms.

The present inventors have found that procathepsin L is a valuable biomarker for ischemia in a subject. The subject may be a critically ill subject, a subject at risk of developing ischemia-related complications, a subject under revalidation or a sportsperson.

The term “critically ill subject” can be used interchangeably herein with the recitations “subject with a condition requiring critical care”, “subject with a critical illness” or “subject with a critical care condition”.

The terms “critically ill”, “critical illness”, “condition which requires critical care”, or “critical care condition” can be used interchangeably herein and generally refer to a condition which is life threatening to the sufferer and may thus result in death within a relatively short period of time such as within hours or days. Such conditions require critical care (e.g. monitoring and treatment) that generally involves close, constant attention by a team of specially trained health professionals. Such care usually takes place in an intensive care unit (ICU), emergency department (ED) or trauma centre. However, care might take place in any appropriate unit which has a similar or equivalent structure and capability as an ICU, ED or trauma centre. Thus, preferred critical conditions for application of the methods of the present invention are conditions requiring admittance to an ICU, ED or a setting which has a similar or equivalent structure and capability such as a trauma centre and preferred patients are ICU patients, ED patients or trauma centre patients.

Such critical care conditions include complications from surgery, life threatening accidents or other life threatening physical trauma or stress; medical shock i.e. a condition when insufficient blood flow reaches body tissues; infections e.g. bacterial, fungal or viral infections; systemic inflammatory response syndrome (SIRS); sepsis; severe sepsis i.e. sepsis with organ dysfunction; septic shock i.e. sepsis with acute circulatory failure; (for sepsis-related definitions see Levy M M, et al., Crit. Care Med, 2003, 31, 1250-56 and the definitions provided by the American College of Chest Physicians and the Society of Critical Care Medicine, Crit. Care Med., 1992, 20:864-874); Acute Respiratory Distress Syndrome (ARDS) defined by pulmonary and systemic inflammation and pulmonary tissue injury (including endothelial and/or epithelial tissue) injury that result in alveolar filling and respiratory failure (Bajwa et al., Crit. Care Med., 2007, 35, 2484-2490); severe pneumonia; respiratory failure particularly acute respiratory failure; respiratory distress; severe chronic obstructive pulmonary disease (COPD); subarachnoidal hemorrhage (SAH); (severe) stroke; asphyxia; neurological conditions; organ dysfunction; single or multi-organ failure (MOF); poisoning and intoxication; severe allergic reactions and anaphylaxis; acute gastrointestinal and abdominal conditions resulting in SIRS; burn injury; acute cerebral hemorrhage or infarction; and any condition for which the patient requires assisted (e.g. mechanical) ventilation. It should be noted that, by their very nature, such conditions which require critical care are serious, severe, life-threatening forms of illness.

Methods of the present invention have been shown to work inter alia in cardiac surgery ICU patients including patients undergoing coronary artery bypass graft (CABG) and valve repair or replacement.

The recitation “subject at risk of developing ischemia-related complications”, as used herein refers to a subject having one or more of the conditions selected from atherosclerosis, diabetes, obesity, ischemic heart disease, chronic heart failure, (history of) heart valve problems, lipid disorders, lipoprotein disorders, and claudicatio. A subject at risk of developing ischemia-related complications may further have any other condition that may lead to ischemic stress.

The recitation “subject under revalidation”, as used herein, refers to a subject undergoing a program, for instance an anaerobic exercise program, in order to improve his health status or health state. For example, the subject under revalidation can be a patient recently discharged from the ICU, or a patient who was or still is at risk of developing ischemia-related complications.

The terms “sportsperson”, “sportsman” or “sportswoman” can be used interchangeably herein and generally refers to a person trained to compete in a sport involving physical strength, speed or endurance. Sportspersons may be professional or amateur.

The inventors further show that procathepsin L, cathepsin L, or a fragment thereof is an important biomarker for diagnosing, predicting, prognosticating and/or monitoring ischemia in a subject, wherein said diagnosis, prediction, prognosis and/or monitoring ischemia comprises assessing the degree of ischemia in the subject,

The term “degree” as used herein, refers to the severity of a disease or condition. For instance and without any limitation, the degree of a disease can be classified according to an ICU scoring system such as the APACHE II system. The term “APACHE II” or “Acute Physiology and Chronic Health Evaluation II” (Knaus et al., 1985, Crit. Care Med., 13(10), 818-29) refers to one of several ICU scoring systems which is applied within 24 hours of admission of a patient to an intensive care unit. An integer score from 0 to 71 is computed based on several measurements, whereby higher scores correspond to more severe disease and a higher risk of death.

The degree of ischemia in a subject may be assessed in accordance with lactate levels. The degree of ischemia may also be assessed as being: (i) no ischemia, ii) low degree of ischemia with reversible or reparable physiological outcome, or (iii) high degree of ischemia with potential irreversible or irreparable physiological damage, morbidity or mortality.

The term “morbidity” generally refers to a diseased state, disability, or poor health due to any cause. The term may be used to refer to the existence of any form of disease, or to the degree that the condition affects the patient. Among critically ill patients, the level of morbidity is often measured by ICU scoring systems such as APACHE II, SAPS II and III, Glasgow Coma scale, PIM2, and SOFA.

The term “mortality” generally refers to the state or condition of being mortal or susceptible to death.

Further provided are the uses and method as defined herein, wherein said diagnosis, prediction, prognosis and/or monitoring ischemia comprises distinguishing subjects with a likely favourable outcome from subjects with ischemia-related complications. The ischemia-related complications can be one or more of acute kidney injury (AKI), cardiogenic shock, myocardial infarction, heart failure, death, amputation or removal of the damaged area, organ or limb, brain infarction and its neurological deficits, and any organ damage or failure.

The term “acute kidney injury” (AKI) or “acute renal failure” (ARF)” generally refers to a rapid loss of kidney function. Acute kidney injury may be staged (classified, graded) into 5 distinct stages using the “RIFLE” (Risk, Injury, Failure, Loss, End-stage renal disease) staging system as set out here below (based on Lameire et al. 2005, Lancet, 365: 417-430):

| | GFR (based on | Urine |
|---|---|---|
| Stage | serum creatinine) criteria | output criteria |
| “Risk” | Serum creatinine increased 1.5 times | <0.5 ml/kg/h for 6 h |
| “Injury” | Serum creatinine increased 2.0 times | <0.5 ml/kg/h for 12 h |
| “Failure” | Serum creatinine increased 3.0 times, | <0.3 ml/kg/h for 24 h |
| | or creatinine >355 mM/l when there | or anuria for 12 h |
| | was an acute rise of >44 mM/l | |
| “Loss” | Persistent acute renal failure >4 weeks | — |
| “End-stage” | End-stage renal disease >3 months | — |

GFR = glomerular filtration rate

Acute kidney injury may also be staged using the “AKIN” (Acute Kidney Injury Network) criteria as set out here below (based on Bagshaw et al. 2008, Nephrol. Dial. Transplant., 23(5): 1569-1574):

| Stage | Serum creatinine criteria | Urine output criteria |
|---|---|---|
| Stage 1 | Increase in serum creatinine ≧26.2 | <0.5 ml/kg/h for ≧6 h |
| | μmol/l or increase to ≧150-199% | |
| | (1.5- to 1.9-fold) from baseline | |
| Stage 2 | Increase in serum creatinine to 200- | <0.5 ml/kg/h for ≧12 h |
| | 299% (>2-2.9 fold) from baseline | |
| Stage 3 | Increase in serum creatinine to ≧300% | <0.3 ml/kg/h ≧24 h |
| | (≧3-fold) from baseline or serum | or anuria ≧12 h |
| | creatinine ≧354 μmol/l with an acute | |
| | rise of at least 44 μmol/l or | |
| | initiation of RRT | |

The term “cardiogenic shock” relates to sustained hypotension with tissue hypoperfusion despite adequate left ventricular filling pressure. Hypoperfusion or low blood pressure can be due to low blood volume, hormonal changes, widening of blood vessels, medicine side effects, anemia, heart & endocrine problems. Cardiogenic shock is caused by the failure of the heart to pump effectively. It can be due to damage to the heart muscle, most often from a large myocardial infarction. Other causes include arrhythmia, cardiomyopathy, cardiac valve problems, ventricular outflow obstruction (i.e. aortic valve stenosis, aortic dissection, systolic anterior motion (SAM) in hypertrophic cardiomyopathy) or ventriculoseptal defects.

The term “myocardial infarction” (MI), also referred to as “acute myocardial infarction” (AMI), commonly known as a heart attack, generally refers to the interruption of blood supply to a part of the heart, causing heart cells to die.

The term “heart failure”, as used herein, encompasses “acute heart failure”. “Heart failure” and “acute heart failure” carry their respective art-established meanings. By means of further guidance, the term “heart failure” as used herein broadly refers to pathological conditions characterised by an impaired diastolic or systolic blood flow rate and thus insufficient blood flow from the ventricle to peripheral organs.

“Acute heart failure” (AHF) or also termed “acute decompensated heart failure” may be defined as the rapid onset of symptoms and signs secondary to abnormal cardiac function, resulting in the need for urgent therapy. AHF can present itself acute de novo (new onset of acute heart failure in a patient without previously known cardiac dysfunction) or as acute decompensation of chronic heart failure.

The cardiac dysfunction may be related to systolic or diastolic dysfunction, to abnormalities in cardiac rhythm, or to preload and afterload mismatch. It is often life threatening and requires urgent treatment. According to established classification, acute heart failure includes several distinct clinical conditions of presenting patients: (I) acute decompensated congestive heart failure, (II) AHF with hypertension/hypertensive crisis, (Ill) AHF with pulmonary oedema, (IVa) cardiogenic shock, or low output syndrome, (IVb) severe cardiogenic shock, (V) high output failure, and (VI) right-sided acute heart failure. For detailed clinical description, classification and diagnosis of AHF, and for summary of further AHF classification systems including the Killip classification, the Forrester classification and the ‘clinical severity’ classification, refer inter alia to Nieminen et al. 2005 (Eur. Heart J, 26: 384-416) and references therein.

The term “death” generally refers to the permanent termination of the biological functions that sustain a living organism.

The term “brain infarction” or “cerebral infarction” generally refers to an ischemic kind of stroke due to a disturbance in the blood vessels supplying blood to the brain.

The term “neurological deficits” or “functional neurological deficits” refers to a variety of symptoms of apparent neurological origin but which current models struggle to explain psychologically or organically.

The term “organ failure” generally refers to a condition where an organ does not perform its expected function. Organ failure relates to organ dysfunction to such a degree that normal homeostasis cannot be maintained without external clinical intervention. Examples of organ failure include without limitation renal failure, (acute) liver failure, heart failure, and respiratory failure.

Further disclosed are the uses and method as defined herein, wherein said diagnosis, prediction, prognosis and/or monitoring ischemia comprises assessing the risk of developing ischemia-related complications in a subject having one or more of the conditions selected from atherosclerosis, diabetes, obesity, ischemic heart disease, chronic heart failure, heart valve problems, lipid disorders, lipoprotein disorders, and claudicatio.

The conditions “atherosclerosis”, “diabetes”, “obesity” “ischemic heart disease”, “chronic heart failure”, “heart valve problems”, “lipid disorders”, “lipoprotein disorders” and “claudicatio” can be understood as is known in the art.

By means of further guidance, the term “atherosclerosis” also known as “arteriosclerotic vascular disease” or “ASVD” generally refers to a condition in which an artery wall thickens as a result of the accumulation of fatty materials such as cholesterol.

The term “diabetes mellitus” or “diabetes” generally refers to a group of metabolic diseases in which a person has high blood sugar, either because the body does not produce enough insulin, or because cells do not respond to the insulin that is produced. There are three main types of diabetes:
(i) Type I diabetes, insulin-dependent diabetes mellitus (IDDM) or juvenile diabetes which results from the body's failure to produce insulin.
(ii) Type II diabetes, non-insulin-dependent diabetes mellitus (NIDDM) or adult-onset diabetes which results from insulin resistance, a condition in which cells fail to use insulin properly, sometimes combined with an absolute insulin deficiency.
(iii) Gestational diabetes: when pregnant women, who have never had diabetes before, have a high blood glucose level during pregnancy. It may precede development of Type II diabetes.

Other forms of diabetes mellitus include congenital diabetes, which is due to genetic defects of insulin secretion, cystic fibrosis-related diabetes, steroid diabetes induced by high doses of glucocorticoids, and several forms of monogenic diabetes. Type II diabetes can develop in obese or metabolic syndrome patients.

The term “obesity” refers to a medical condition in which excess body fat has accumulated to the extent that it may have an adverse effect on health, leading to reduced life expectancy and/or increased health problems. The “body mass index” (BMI) is a heuristic indicator for human body fat based on an individual's weight and height. The body mass index is defined as the individual's body weight divided by the square of his height. People are defined as overweight or pre-obese if their BMI is between 25 and 30 kg/m², and obese when their BMI is greater than 30 kg/m².

The terms “ischemic heart disease” (1HD) or “myocardial ischemia” generally refers to a disease characterized by ischemia of the heart muscle, usually due to coronary artery disease i.e. atherosclerosis of the coronary arteries. Symptoms of stable ischemic heart disease include angina and decreased exercise tolerance.

The term “chronic heart failure” (CHF) generally refers to a case of heart failure that progresses so slowly that various compensatory mechanisms work to bring the disease into equilibrium. Common clinical symptoms of CHF include inter alia any one or more of breathlessness, diminishing exercise capacity, fatigue, lethargy and peripheral oedema. Other less common symptoms include any one or more of palpitations, memory or sleep disturbance and confusion, and usually co-occur with one or more of the above recited common symptoms.

The recitation “heart valve problems” relates to a disease or condition wherein there is a problem with one or more of the four valves of the heart. Heart valve problems include valve stenosis and valve regurgitation. Valve stenosis refers to the condition wherein a valve becomes narrow and the blood can not easily flow into the next chamber or blood vessel. Valve regurgitation refers to the condition wherein a valve does not close properly and becomes leaky causing blood to flow in the wrong direction.

The term “lipid disorders” generally refers to disorders or abnormalities in lipid metabolism or storage. An exemplary lipid disorder is hypercholesterolemia. The term “hypercholesterolemia” refers to the presence of high levels of cholesterol in the blood.

The term “lipoprotein disorders” relates to hypolipidemia or hypolipoprotinemia (decreased levels of lipids and/or lipoproteins in the blood) and hyperlipidemia or hyperlipoproteinemia (elevated levels of lipids and/or lipoproteins in the blood).

The term “claudicatio” generally refers to a peripheral vascular disease causing an impairment in walking, or a painful, aching, cramping, uncomfortable, or tired feeling in the legs that occurs during walking and that is relieved by rest. The term “peripheral vascular disease” (PVD), commonly referred to as peripheral arterial disease (PAD) or peripheral artery occlusive disease (PAOD), refers to the obstruction of large arteries not within the coronary, aortic arch vasculature, or brain. PVD can result from atherosclerosis, inflammatory processes leading to stenosis, an embolism, or thrombus formation.

Also provided are the uses and method as defined herein, wherein said monitoring ischemia comprises optimizing the physical training program of a subject. Such use advantageously allows one to monitor and improve the health state of the subject.

The terms “heath”, “health state”, or “health status” can be used interchangeably herein and generally refer to a level of functional efficiency, metabolic efficiency or functional and metabolic efficiency of a living being. The term “good health” may generally refer to being free from illness, injury or pain. The term “poor health” may generally refer to having one or more of illness, injury or pain.

The terms “predicting” or “prediction”, “diagnosing” or “diagnosis” and “prognosticating” or “prognosis” are commonplace and well-understood in medical and clinical practice. It shall be understood that the phrase “a method for predicting, diagnosing and/or prognosticating” a given disease or condition may also be interchanged with phrases such as “a method for prediction, diagnosis and/or prognosis” of said disease or condition or “a method for making (or determining or establishing) a prediction, diagnosis and/or prognosis” of said disease or condition, or the like.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as described herein in a subject may particularly mean that the subject has a ‘negative’ prediction of such, i.e., that the subject's risk of having such is not significantly increased vis-à-vis a control subject or subject population.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

The terms “prognosticating” or “prognosis” generally refer to an anticipation on the progression of a disease or condition and the prospect (e.g., the probability, duration, and/or extent) of recovery.

A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period.

A poor prognosis of the diseases or conditions as taught herein may generally encompass anticipation of a substandard recovery and/or unsatisfactorily slow recovery, or to substantially no recovery or even further worsening of such.

The term “subject” or “patient” as used herein typically denotes humans, but may also encompass reference to non-human animals, preferably warm-blooded animals, more preferably mammals, such as, e.g., non-human primates, rodents, canines, felines, equines, ovines, porcines, and the like.

The terms “sample” or “biological sample” as used herein include any biological specimen obtained from a subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. Preferred samples may include ones comprising the protein procathepsin L, cathepsin L or a fragment thereof in detectable quantities. In preferred embodiments, the sample may be whole blood or a fractional component thereof such as, e.g., plasma, serum, or a cell pellet. Preferably the sample is readily obtainable by minimally invasive methods, allowing removal or isolation of said sample from the subject. Samples may also include tissue samples and biopsies, tissue homogenates and the like. Preferably, the sample used to detect procathepsin L, cathepsin L or a fragment thereof is serum. Equally preferred, the sample used to detect procathepsin L, cathepsin L or a fragment thereof is plasma.

The term “serum” refers to the component of blood that is neither a blood cell nor a clotting factor; the term refers to the blood plasma with the fibrinogens removed.

The term “plasma” defines the colourless watery fluid of the blood that contains no cells, but in which the blood cells (erythrocytes, leukocytes, thrombocytes, etc.) are suspended, containing nutrients, sugars, proteins, minerals, enzymes, etc.

A molecule or analyte such as a protein, polypeptide or peptide, or a group of two or more molecules or analytes such as two or more proteins, polypeptides or peptides, is “measured” in a sample when the presence or absence, quantity and/or activity of said molecule or analyte or of said group of molecules or analytes is detected or determined in the sample, preferably substantially to the exclusion of other molecules and analytes.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities, or first and second activities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need first to convert the readouts to absolute values of the respective parameters.

The terms “activity”, “enzymatic activity” and “biological activity” can be used interchangeably herein and are generally well-understood in the art. The terms as used herein may particularly refer to an absolute activity of a molecule or analyte in a sample, or to a relative activity of a molecule or analyte in a sample, i.e., relative to another value such as relative to a reference value as taught herein, or to a range of values indicating a base-line activity of the biomarker. These values or ranges can be obtained from a single patient or from a group of patients.

An absolute activity of a molecule or analyte in a sample may be advantageously expressed as moles of substrate converted (or product produced) per unit time, or more commonly as the concentration of substrate converted per unit time, e.g. mol s⁻¹ or M s⁻¹.

A relative activity of a molecule or analyte in a sample may be advantageously expressed as an increase or decrease or as a fold-increase or fold-decrease relative to said another value, such as relative to a reference value as taught herein.

As used herein, the term “procathepsin L” refers to the pro-form of cathepsin L or the preproprotein of cathepsin L. The term “procathepsin L” as used herein encompasses the (pre)-proprotein of cathepsin L as well as fragments thereof.

The term “cathepsin L”, corresponds to the protein commonly known as “cathepsin L1”, also known as “CATL”, “CTSL” or “CTSL1”, i.e. the proteins and polypeptides commonly known under these designations in the art. The terms encompass such proteins and polypeptides of any organism where found, and particularly of animals, preferably vertebrates, more preferably mammals, including humans and non-human mammals, even more preferably of humans. The terms particularly encompass such proteins and polypeptides with a native sequence, i.e., ones of which the primary sequence is the same as that of procathepsin L or cathepsin L found in or derived from nature. A skilled person understands that native sequences of procathepsin L or cathepsin L may differ between different species due to genetic divergence between such species. Moreover, the native sequences of procathepsin L or cathepsin L may differ between or within different individuals of the same species due to normal genetic diversity (variation) within a given species. Also, the native sequences of procathepsin L or cathepsin L may differ between or even within different individuals of the same species due to post-transcriptional or post-translational modifications. Accordingly, all procathepsin L or cathepsin L sequences found in or derived from nature are considered “native”. The terms encompass procathepsin L or cathepsin L proteins and polypeptides when forming a part of a living organism, organ, tissue or cell, when forming a part of a biological sample, as well as when at least partly isolated from such sources. The terms also encompass proteins and polypeptides when produced by recombinant or synthetic means.

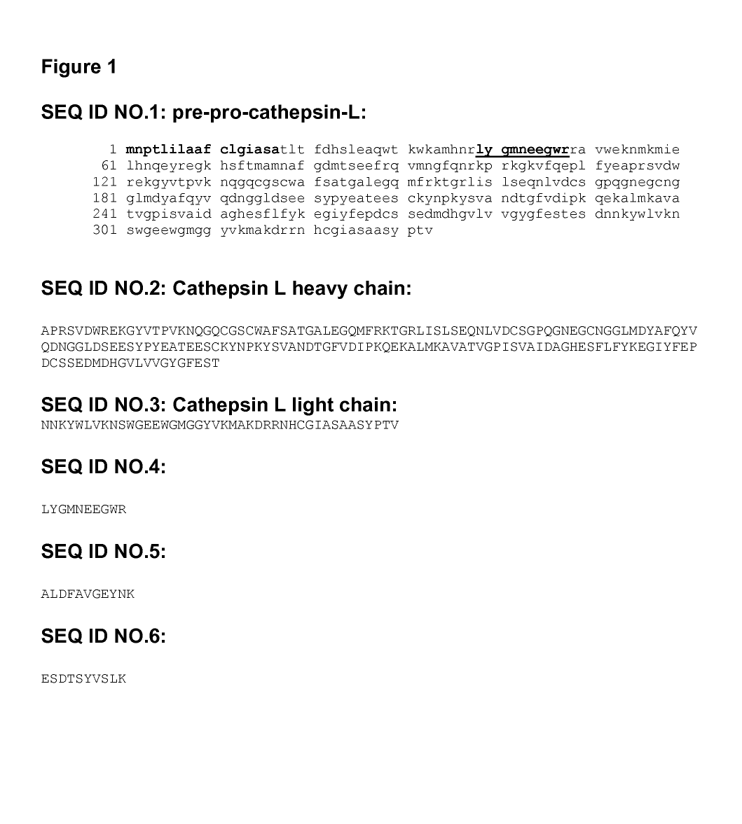

Exemplary procathepsin L includes, without limitation, human pre-procathepsin L having primary amino acid sequence as annotated under NCBI Genbank (http://www.ncbi.nlm.nih.gov/) accession number NP_{—}001903 (sequence version 1), comprising 333 amino acids as reproduced in FIG. 1 (SEQ ID NO: 1) or as annotated under NCBI Genbank accession number NP_{—}666023 (sequence version 1), comprising 333 amino acids. During maturation, the signal peptide of the protein (bold in FIG. 1) is removed to form the inactive procathepsin L, which also falls within the definition of procathepsin L herein. Subsequently, the activation peptide is removed from the inactive procathepsin L and heavy (SEQ ID NO: 2) and light chains (SEQ ID NO:3) are produced which are consequently linked by disulfide bridges to form the biological active cathepsin L protein. A skilled person can also appreciate that said sequences are the precursor of cathepsin L and may include parts which are processed away from mature cathepsin L. Exemplary cathepsin L includes human cathepsin L which is a dimer composed of disulfide-linked heavy and light chains, both produced from a single protein precursor, i.e. procathepsin L as defined above. The term “cathepsin L” as used herein encompasses full-length cathepsin L as well as fragments thereof.

In an embodiment the circulating procathepsin L, cathepsin L or fragments thereof, e.g., secreted form circulating in the blood plasma, may be detected, as opposed to the cell-bound or cell-confined procathepsin L, cathepsin L or fragments thereof.

The reference herein to procathepsin L, cathepsin L or a fragment thereof may thus also encompass fragments of procathepsin L or cathepsin L. Hence, the reference herein to measuring procathepsin L, cathepsin L or a fragment thereof, or to measuring the quantity of procathepsin L, cathepsin L or a fragment thereof, may encompass measuring the protein or polypeptide of procathepsin L, cathepsin L or a fragment thereof, such as, e.g., measuring the mature, active and/or the processed soluble/secreted form (e.g. plasma circulating form) of procathepsin L or cathepsin L and/or measuring one or more fragments thereof. For example, procathepsin L, cathepsin L or a fragment thereof may be measured collectively, such that the measured quantity corresponds to the sum amounts of the collectively measured species, by for example using a binding molecule that binds the heavy or light chains of cathepsin L. In another example, procathepsin L, cathepsin L and/or one or more fragments thereof may each be measured individually. Preferably, said fragment of procathepsin L or cathepsin L is a plasma circulating form of procathepsin L or cathepsin L. The expression “plasma circulating form of procathepsin L or cathepsin L” or shortly “circulating form” encompasses all procathepsin L or cathepsin L proteins or fragments thereof that circulate in the plasma, i.e., are not cell-bound or membrane-bound.

Without wanting to be bound by any theory, such circulating forms may be derived from the full-length procathepsin L or cathepsin L protein through natural processing, or may result from known degradation processes occurring in said sample. In certain situations, the circulating form may also be the full-length procathepsin L or cathepsin L protein, which is found to be circulating in the plasma. Said “circulating form” may thus be any procathepsin L or cathepsin

L protein or any processed soluble form of procathepsin L or cathepsin L or fragments of either one, that is circulating in the sample, i.e. which is not bound to a cell- or membrane fraction of said sample.

The peptide detected in the samples of the subjects according to the uses or methods as described herein is situated in the activation peptide part of procathepsin L (FIG. 1, bold underlined, SEQ ID NO:4).

Unless otherwise apparent from the context, reference herein to any protein, polypeptide or peptide encompasses such from any organism where found, and particularly preferably from animals, preferably vertebrates, more preferably mammals, including humans and non-human mammals, even more preferably from humans.

Further, unless otherwise apparent from the context, reference herein to any protein, polypeptide or peptide and fragments thereof may generally also encompass modified forms of said protein, polypeptide or peptide and fragments such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

In an embodiment, procathepsin L, cathepsin L or a fragment thereof, or other biomarkers as employed herein and fragments thereof, may be human, i.e., their primary sequence may be the same as a corresponding primary sequence of or present in a naturally occurring human peptides, polypeptides or proteins. Hence, the qualifier “human” in this connection relates to the primary sequence of the respective proteins, polypeptides, peptides or fragments, rather than to their origin or source. For example, such proteins, polypeptides, peptides or fragments may be present in or isolated from samples of human subjects or may be obtained by other means (e.g., by recombinant expression, cell-free translation or non-biological peptide synthesis).

The term “fragment” of a protein, polypeptide or peptide generally refers to N-terminally and/or C-terminally deleted or truncated forms of said protein, polypeptide or peptide. The term encompasses fragments arising by any mechanism, such as, without limitation, by alternative translation, exo- and/or endo-proteolysis and/or degradation of said protein or polypeptide, such as, for example, in vivo or in vitro, such as, for example, by physical, chemical and/or enzymatic proteolysis. Without limitation, a fragment of a protein, polypeptide or peptide may represent at least about 5%, or at least about 10%, e.g., ≧20%, ≧30% or ≧40%, such as ≧50%, e.g., ≧60%, ≧70% or ≧80%, or even ≧90% or ≧95% of the amino acid sequence of said protein, polypeptide or peptide.

For example, a fragment may include a sequence of ≧5 consecutive amino acids, or ≧10 consecutive amino acids, or ≧20 consecutive amino acids, or ≧30 consecutive amino acids, e.g., ≧40 consecutive amino acids, such as for example ≧50 consecutive amino acids, e.g., ≧60, ≧70, ≧80, ≧90, ≧100, ≧200, ≧300, ≧400, ≧500 or ≧600 consecutive amino acids of the corresponding full length protein.

In an embodiment, a fragment may be N-terminally and/or C-terminally truncated by between 1 and about 20 amino acids, such as, e.g., by between 1 and about 15 amino acids, or by between 1 and about 10 amino acids, or by between 1 and about 5 amino acids, compared to the corresponding mature, full-length protein or its soluble or plasma circulating form. By means of example, proBNP, NTproBNP and BNP fragments useful as biomarkers are disclosed in WO 2004/094460.

In an embodiment, fragments of a given protein, polypeptide or peptide may be achieved by in vitro proteolysis of said protein, polypeptide or peptide to obtain advantageously detectable peptide(s) from a sample. For example, such proteolysis may be effected by suitable physical, chemical and/or enzymatic agents, e.g., proteinases, preferably endoproteinases, i.e., protease cleaving internally within a protein, polypeptide or peptide chain. A non-limiting list of suitable endoproteinases includes serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases. Exemplary non-limiting endoproteinases include trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain). Further known or yet to be identified enzymes may be used; a skilled person can choose suitable protease(s) on the basis of their cleavage specificity and frequency to achieve desired peptide forms. Preferably, the proteolysis may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, trypsin in solution, trypsin immobilised to a solid support, etc. Trypsin is particularly useful, inter alia due to high specificity and efficiency of cleavage. The invention also contemplates the use of any trypsin-like protease, i.e., with a similar specificity to that of trypsin. Otherwise, chemical reagents may be used for proteolysis. For example, CNBr can cleave at Met; BNPS-skatole can cleave at Trp. The conditions for treatment, e.g., protein concentration, enzyme or chemical reagent concentration, pH, buffer, temperature, time, can be determined by the skilled person depending on the enzyme or chemical reagent employed.

Also provided is thus an isolated fragment of procathepsin L or cathepsin L as defined here above. Such fragments may give useful information about the presence and quantity of procathepsin L or cathepsin L in biological samples, whereby the detection of said fragments is of interest. Hence, the herein disclosed fragments of procathepsin L or cathepsin L are useful biomarkers. A preferred procathepsin L or cathepsin L fragment may comprise, consist essentially of or consist of the sequence as set forth in any one of the sequences defined by SEQ ID NOs: 1 to 4.

The term “isolated” with reference to a particular component (such as for instance, a protein, polypeptide, peptide or fragment thereof) generally denotes that such component exists in separation from—for example, has been separated from or prepared in separation from—one or more other components of its natural environment. For instance, an isolated human or animal protein, polypeptide, peptide or fragment exists in separation from a human or animal body where it occurs naturally.

The term “isolated” as used herein may preferably also encompass the qualifier “purified”. As used herein, the term “purified” with reference to protein(s), polypeptide(s), peptide(s) and/or fragment(s) thereof does not require absolute purity. Instead, it denotes that such protein(s), polypeptide(s), peptide(s) and/or fragment(s) is (are) in a discrete environment in which their abundance (conveniently expressed in terms of mass or weight or concentration) relative to other proteins is greater than in a biological sample. A discrete environment denotes a single medium, such as for example a single solution, gel, precipitate, lyophilisate, etc. Purified peptides, polypeptides or fragments may be obtained by known methods including, for example, laboratory or recombinant synthesis, chromatography, preparative electrophoresis, centrifugation, precipitation, affinity purification, etc.

Purified protein(s), polypeptide(s), peptide(s) and/or fragment(s) may preferably constitute by weight ≧10%, more preferably ≧50%, such as ≧60%, yet more preferably ≧70%, such as ≧80%, and still more preferably ≧90%, such as ≧95%, ≧96%, ≧97%, ≧98%, ≧99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Also, purity of peptides or polypeptides may be determined by SDS-PAGE under reducing or non-reducing conditions using Coomassie blue or, preferably, silver stain.

Further disclosed are isolated procathepsin L, cathepsin L or a fragment thereof as taught herein comprising a detectable label. This facilitates ready detection of such fragments. The term “label” as used throughout this specification refers to any atom, molecule, moiety or biomolecule that can be used to provide a detectable and preferably quantifiable read-out or property, and that can be attached to or made part of an entity of interest, such as a peptide or polypeptide or a specific-binding agent. Labels may be suitably detectable by mass spectrometric, spectroscopic, optical, colorimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish phosphatise or alkaline phosphatise as commonly used in immunoassays); binding moieties such as biotin-streptavidin; haptens such as digoxigenin; luminogenic, phosphorescent or fluorogenic moieties; mass tags; and fluorescent dyes alone or in combination with moieties that can suppress or shift emission spectra by fluorescence resonance energy transfer (FRET).

For example, the label may be a mass-altering label. Preferably, a mass-altering label may involve the presence of a distinct stable isotope in one or more amino acids of the peptide vis-à-vis its corresponding non-labelled peptide. Mass-labelled peptides are particularly useful as positive controls, standards and calibrators in mass spectrometry applications. In particular, peptides including one or more distinct isotopes are chemically alike, separate chromatographically and electrophoretically in the same manner and also ionise and fragment in the same way. However, in a suitable mass analyser such peptides and optionally select fragmentation ions thereof will display distinguishable m/z ratios and can thus be discriminated. Examples of pairs of distinguishable stable isotopes include H and D, ¹²C and ¹³C, ¹⁴N and ¹⁵N or ¹⁶O and ¹⁸O. Usually, peptides and proteins of biological samples analysed in the present invention may substantially only contain common isotopes having high prevalence in nature, such as for example H, ¹²C, ¹⁴N and ¹⁶O. In such case, the mass-labelled peptide may be labelled with one or more uncommon isotopes having low prevalence in nature, such as for instance D, ¹³C, ¹⁵N and/or ¹⁸O. It is also conceivable that in cases where the peptides or proteins of a biological sample would include one or more uncommon isotopes, the mass-labelled peptide may comprise the respective common isotope(s).

Isotopically-labelled synthetic peptides may be obtained inter alia by synthesising or recombinantly producing such peptides using one or more isotopically-labelled amino acid substrates, or by chemically or enzymatically modifying unlabelled peptides to introduce thereto one or more distinct isotopes. By means of example and not limitation, D-labelled peptides may be synthesised or recombinantly produced in the presence of commercially available deuterated L-methionine CH₃—S—CD₂CD₂-CH(NH₂)—COOH or deuterated arginine H₂NC(═NH)—NH—(CD₂)₃—CD(NH₂)—COOH. It shall be appreciated that any amino acid of which deuterated or ¹⁵N— or ¹³C-containing forms exist may be considered for synthesis or recombinant production of labelled peptides. In another non-limiting example, a peptide may be treated with trypsin in H₂ ¹⁶O or H₂ ¹⁸O, leading to incorporation of two oxygens (¹⁶O or ¹⁸O, respectively) at the COOH-termini of said peptide (e.g., US 2006/105415).

Accordingly, also contemplated is the use of procathepsin L, cathepsin L or a fragment thereof as taught herein, optionally comprising a detectable label, as (positive) controls, standards or calibrators in qualitative or quantitative detection assays (measurement methods) of procathepsin L, cathepsin L or a fragment thereof, and particularly in such methods as taught herein in subjects. The proteins, polypeptides or peptides may be supplied in any form, inter alia as precipitate, vacuum-dried, lyophilisate, in solution as liquid or frozen, or covalently or non-covalently immobilised on solid phase, such as for example, on solid chromatographic matrix or on glass or plastic or other suitable surfaces (e.g., as a part of peptide arrays and microarrays). The peptides may be readily prepared, for example, isolated from natural sources, or prepared recombinantly or synthetically.

Further disclosed are binding agents capable of specifically binding to any one or more of the isolated fragments of procathepsin L or cathepsin L as taught herein. Also disclosed are binding agents capable of specifically binding to only one of isolated fragments of procathepsin L or cathepsin L as taught herein. Binding agents as intended throughout this specification may include inter alia an antibody, aptamer, spiegelmer (L-aptamer), photoaptamer, protein, peptide, peptidomimetic or a small molecule.

A binding agent may be capable of binding both the plasma circulating form and the cell-bound or retained from of procathepsin L or cathepsin L. Preferably, a binding agent may be capable of specifically binding or detecting the plasma circulating form of procathepsin L or cathepsin L.

The term “specifically bind” as used throughout this specification means that an agent (denoted herein also as “specific-binding agent”) binds to one or more desired molecules or analytes, such as to one or more proteins, polypeptides or peptides of interest or fragments thereof substantially to the exclusion of other molecules which are random or unrelated, and optionally substantially to the exclusion of other molecules that are structurally related. The term “specifically bind” does not necessarily require that an agent binds exclusively to its intended target(s). For example, an agent may be said to specifically bind to protein(s) polypeptide(s), peptide(s) and/or fragment(s) thereof of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Preferably, the agent may bind to its intended target(s) with affinity constant (K_{A}) of such binding K_{A}≧1×10⁶ M⁻¹, more preferably K_{A}≧1×10⁷ M⁻¹, yet more preferably K_{A}≧1×10⁸ M⁻¹, even more preferably K_{A}≧1×10⁹ M⁻¹, and still more preferably K_{A}≧1×10¹⁰ M⁻¹ or K_{A}≧1×10¹¹ M⁻¹, wherein K_{A}=[SBA_T]/[SBA][T], SBA denotes the specific-binding agent, T denotes the intended target. Determination of K_{A} can be carried out by methods known in the art, such as for example, using equilibrium dialysis and Scatchard plot analysis.

Specific binding agents as used throughout this specification may include inter alia an antibody, aptamer, spiegelmer (L-aptamer), photoaptamer, protein, peptide, peptidomimetic or a small molecule.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), lama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

The term “aptamer” refers to single-stranded or double-stranded oligo-DNA, oligo-RNA or oligo-DNA/RNA or any analogue thereof that can specifically bind to a target molecule such as a peptide. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Norwell 1995 (Trends Biotechnol 13: 132-134). Aptamers generally are built up out of naturally occurring D-ribonucleotides. Recently, so called “spiegelmers” have been developed, which are built up out of non-natural “inverse” L-ribonucleotides, which are not easily degradable by the molecular machinery of living cells and hence have a longer half-life.

The term “small molecule” refers to compounds, preferably organic compounds, with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

Hence, also disclosed are methods for immunising animals, e.g., non-human animals such as laboratory or farm, animals using (i.e., using as the immunising antigen) procathepsin L, cathepsin L or a fragment thereof, optionally attached to a presenting carrier. Immunisation and preparation of antibody reagents from immune sera is well-known per se and described in documents referred to elsewhere in this specification. The animals to be immunised may include any animal species, preferably warm-blooded species, more preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Immune sera obtained or obtainable by immunisation as taught herein may be particularly useful for generating antibody reagents that specifically bind to one or more of the herein disclosed fragments of procathepsin L or cathepsin L.

Further disclosed are methods for selecting specific-binding agents which bind (a) one or more of the herein disclosed fragments of procathepsin L or cathepsin L, substantially to the exclusion of (b) procathepsin L, cathepsin L and/or other fragments thereof. Conveniently, such methods may be based on subtracting or removing binding agents which cross-react or cross-bind the non-desired procathepsin L or cathepsin L molecules under (b). Such subtraction may be readily performed as known in the art by a variety of affinity separation methods, such as affinity chromatography, affinity solid phase extraction, affinity magnetic extraction, etc.

Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of procathepsin L, cathepsin L and/or fragments thereof and optionally of the one or more other biomarkers or fragments thereof in samples (any molecules or analytes of interest to be so-measured in samples, including procathepsin L, cathepsin L and fragments thereof, may be herein below referred to collectively as biomarkers).

For example, such methods may include immunoassay methods, mass spectrometry analysis methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding agent, commonly an antibody, and the molecule(s) or analyte(s) of interest. Immunoassay technologies include without limitation direct ELISA (enzyme-linked immunosorbent assay), indirect ELISA, sandwich ELISA, competitive ELISA, multiplex ELISA, radioimmunoassay (RIA), ELISPOT technologies, and other similar techniques known in the art. Principles of these immunoassay methods are known in the art, for example John R. Crowther, “The ELISA Guidebook”, 1st ed., Humana Press 2000, ISBN 0896037282.

By means of further explanation and not limitation, direct ELISA employs a labelled primary antibody to bind to and thereby quantify target antigen in a sample immobilised on a solid support such as a microwell plate. Indirect ELISA uses a non-labelled primary antibody which binds to the target antigen and a secondary labelled antibody that recognises and allows to quantify the antigen-bound primary antibody. In sandwich ELISA the target antigen is captured from a sample using an immobilised ‘capture’ antibody which binds to one antigenic site within the antigen, and subsequent to removal of non-bound analytes the so-captured antigen is detected using a ‘detection’ antibody which binds to another antigenic site within said antigen, where the detection antibody may be directly labelled or indirectly detectable as above. Competitive ELISA uses a labelled ‘competitor’ that may either be the primary antibody or the target antigen. In an example, non-labelled immobilised primary antibody is incubated with a sample, this reaction is allowed to reach equilibrium, and then labelled target antigen is added. The latter will bind to the primary antibody wherever its binding sites are not yet occupied by non-labelled target antigen from the sample. Thus, the detected amount of bound labelled antigen inversely correlates with the amount of non-labelled antigen in the sample. Multiplex ELISA allows simultaneous detection of two or more analytes within a single compartment (e.g., microplate well) usually at a plurality of array addresses (see, for example, Nielsen & Geierstanger 2004. J Immunol Methods 290: 107-20 and Ling et al. 2007. Expert Rev Mol Diagn 7: 87-98 for further guidance). As appreciated, labelling in ELISA technologies is usually by enzyme (such as, e.g., horse-radish peroxidase) conjugation and the end-point is typically colorimetric, chemiluminescent or fluorescent, magnetic, piezo electric, pyroelectric and other.

Radioimmunoassay (RIA) is a competition-based technique and involves mixing known quantities of radioactively-labelled (e.g., ¹²⁵I- or ¹³¹I-labelled) target antigen with antibody to said antigen, then adding non-labelled or ‘cold’ antigen from a sample and measuring the amount of labelled antigen displaced (see, e.g., “An Introduction to Radioimmunoassay and Related Techniques”, by Chard T, ed., Elsevier Science 1995, ISBN 0444821198 for guidance).

Generally, any mass spectrometric (MS) techniques that can obtain precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ⁿ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; and APPI-(MS)ⁿ. Peptide ion fragmentation in tandem MS (MS/MS) arrangements may be achieved using manners established in the art, such as, e.g., collision induced dissociation (CID). Detection and quantification of biomarkers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods described herein below.

Chromatography can also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably HPLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC(NP-HPLC), reversed phase HPLC(RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like.

Chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The various aspects and embodiments taught herein may further rely on comparing the quantity and/or activity of procathepsin L, cathepsin L or a fragment thereof, as defined herein, measured in samples with reference values of the quantity and/or activity of procathepsin L, cathepsin L or a fragment thereof, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values may represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying degree or severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such different between values or profiles being compared. A comparison may include a visual inspection, an arithmetical or statistical comparison of measurements. Such statistical comparisons include, but are not limited to, applying a rule. If the values or biomarker profiles comprise at least one standard, the comparison to determine a difference in said values or biomarker profiles may also include measurements of these standards, such that measurements of the biomarker are correlated to measurements of the internal standards.

Reference values for the quantity and/or activity of procathepsin L, cathepsin L or a fragment thereof may be established according to known procedures previously employed for other biomarkers.

For example, a reference value of the quantity and/or activity of procathepsin L, cathepsin L or a fragment thereof for a particular prediction, diagnosis and/or prognosis of given disease or condition as taught herein may be established by determining the quantity and/or activity of procathepsin L, cathepsin L or a fragment thereof in sample(s) from one individual or from a population of individuals characterised by said particular prediction, diagnosis and/or prognosis of said disease or condition (i.e., for whom said prediction, diagnosis and/or prognosis of ischemia holds true). Such population may comprise without limitation 2, 10, 100, or even several hundreds or more individuals.

Hence, by means of an illustrative example, reference values of the quantity and/or activity of procathepsin L, cathepsin L or a fragment thereof for the diagnoses of a given disease or condition as taught herein vs. no such disease or condition may be established by determining the quantity and/or activity of procathepsin L, cathepsin L or a fragment thereof in sample(s) from one individual or from a population of individuals diagnosed (e.g., based on other adequately conclusive means, such as, for example, clinical signs and symptoms, etc.) as, respectively, having or not having said disease or condition.

In an embodiment, reference value(s) as intended herein may convey absolute quantities and/or activities of procathepsin L, cathepsin L or a fragment thereof. In another embodiment, the quantity and/or activity of procathepsin L, cathepsin L or a fragment thereof in a sample from a tested subject may be determined directly relative to the reference value (e.g., in terms of increase or decrease, or fold-increase or fold-decrease). Advantageously, this may allow the comparison of the quantity and/or activity of procathepsin L, cathepsin L or a fragment thereof in the sample from the subject with the reference value (in other words to measure the relative quantity and/or activity of procathepsin L, cathepsin L or a fragment thereof in the sample from the subject vis-à-vis the reference value) without the need first to determine the respective absolute quantities of procathepsin L, cathepsin L or a fragment thereof.

The expression level or presence of a biomarker in a sample of a patient may sometimes fluctuate, i.e. increase or decrease significantly without change (appearance of, worsening or improving of) symptoms. In such an event, the marker change precedes the change in symptoms and becomes a more sensitive measure than symptom change. Therapeutic intervention can be initiated earlier and be more effective than waiting for deteriorating symptoms. Early intervention for instance in patients at risk of developing ischemia-related complications may be carried out safely at home, which is a major improvement from treating critically ill patients in the emergency department or intensive care unit.

Measuring the level of procathepsin L, cathepsin L or a fragment thereof of the same patient at different time points may in such a case thus enable the continuous monitoring of the status of the patient and may lead to prediction of worsening or improvement of the patient's condition with regard to a given disease or condition as taught herein. A home or clinical test kit or device as indicated herein may be used for this continuous monitoring. One or more reference values or ranges of levels of procathepsin L, cathepsin L or a fragment thereof linked to a certain disease state (e.g. ischemia or no ischemia) for such a test may e.g. be determined beforehand or during the monitoring process over a certain period of time in said subject. Alternatively, these reference values or ranges may be established through data sets of several patients with highly similar disease phenotypes, e.g. from healthy subjects or subjects not having the disease or condition of interest. A sudden deviation of the levels of procathepsin L, cathepsin L or a fragment thereof from said reference value or range may predict the worsening of the condition of the patient (e.g. at home or in the clinic) before the (often severe) symptoms actually can be felt or observed.

Also disclosed is thus a method or algorithm for determining a significant change in the level of procathepsin L, cathepsin L or a fragment thereof as a marker in a certain patient, which is indicative for change (worsening or improving) in clinical status. In addition, the invention allows establishing the diagnosis that the subject is recovering or has recovered from a given disease or condition as taught herein.

In an embodiment the present methods may include a step of establishing such reference value(s). In an embodiment, the present kits and devices may include means for establishing a reference value of the quantity and/or activity of procathepsin L, cathepsin L or a fragment thereof for a particular prediction, diagnosis and/or prognosis of a given disease or condition as taught herein. Such means may for example comprise one or more samples (e.g., separate or pooled samples) from one or more individuals characterised by said particular prediction, diagnosis and/or prognosis of said disease or condition.

The various aspects and embodiments taught herein may further entail finding a deviation or no deviation between the quantity and/or activity of procathepsin L, cathepsin L or a fragment thereof measured in a sample from a subject and a given reference value.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD, or ±1×SE or ±2×SE). Deviation may also refer to a value falling outside of a reference range defined by values in a given population (for example, outside of a range which comprises ≧40%, ≧50%, ≧60%, ≧70%, ≧75% or ≧80% or ≧85% or ≧90% or ≧95% or even ≧100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction, diagnosis and/or prognosis methods, e.g., sensitivity and/or specificity of at least 50%, or at least 60%, or at least 70%, or at least 80%, or at least 85%, or at least 90%, or at least 95%.

For example, in an embodiment, an elevated quantity and/or activity of procathepsin L, cathepsin L or a fragment thereof in the sample from the subject—preferably at least about 1.1-fold elevated, or at least about 1.2-fold elevated, more preferably at least about 1.3-fold elevated, even more preferably at least about 1.4-fold elevated, yet more preferably at least about 1.5-fold elevated, such as between about 1.1-fold and 3-fold elevated or between about 1.5-fold and 2-fold elevated—compared to a reference value representing the prediction or diagnosis of no given disease or condition as taught herein or representing a good prognosis for said disease or condition indicates that the subject has or is at risk of having said disease or condition or indicates a poor prognosis for the disease or condition in the subject.

When a deviation is found between the quantity and/or activity of procathepsin L, cathepsin L or a fragment thereof in a sample from a subject and a reference value representing a certain prediction, diagnosis and/or prognosis of a given disease or condition as taught herein, said deviation is indicative of or may be attributed to the conclusion that the prediction, diagnosis and/or prognosis of said disease or condition in said subject is different from that represented by the reference value.

When no deviation is found between the quantity and/or activity of procathepsin L, cathepsin L or a fragment thereof in a sample from a subject and a reference value representing a certain prediction, diagnosis and/or prognosis of a given disease or condition as taught herein, the absence of such deviation is indicative of or may be attributed to the conclusion that the prediction, diagnosis and/or prognosis of said disease or condition in said subject is substantially the same as that represented by the reference value.

The above considerations apply analogously to biomarker profiles.

When two or more different biomarkers are determined in a subject, their respective presence, absence and/or quantity may be together represented as a biomarker profile, the values for each measured biomarker making a part of said profile. As used herein, the term “profile” includes any set of data that represents the distinctive features or characteristics associated with a condition of interest, such as with a particular prediction, diagnosis and/or prognosis of a given disease or condition as taught herein. The term generally encompasses inter alia nucleic acid profiles, such as for example genotypic profiles (sets of genotypic data that represents the genotype of one or more genes associated with a condition of interest), gene copy number profiles (sets of gene copy number data that represents the amplification or deletion of one or more genes associated with a condition of interest), gene expression profiles (sets of gene expression data that represents the mRNA levels of one or more genes associated with a condition of interest), DNA methylation profiles (sets of methylation data that represents the DNA methylation levels of one or more genes associated with a condition of interest), as well as protein, polypeptide or peptide profiles, such as for example protein expression profiles (sets of protein expression data that represents the levels of one or more proteins associated with a condition of interest), protein activation profiles (sets of data that represents the activation or inactivation of one or more proteins associated with a condition of interest), protein modification profiles (sets of data that represents the modification of one or more proteins associated with a condition of interest), protein cleavage profiles (sets of data that represent the proteolytic cleavage of one or more proteins associated with a condition of interest), as well as any combinations thereof.

Biomarker profiles may be created in a number of ways and may be the combination of measurable biomarkers or aspects of biomarkers using methods such as ratios, or other more complex association methods or algorithms (e.g., rule-based methods). A biomarker profile comprises at least two measurements, where the measurements can correspond to the same or different biomarkers. A biomarker profile may also comprise at least three, four, five, 10, 20, or more measurements. In one embodiment, a biomarker profile comprises hundreds, or even thousands, of measurements.

Hence, for example, distinct reference profiles may represent the prediction of a risk (e.g., an abnormally elevated risk) of having a given disease or condition vs. the prediction of no or normal risk of having said disease or condition. In another example, distinct reference profiles may represent predictions of differing degrees of risk of having said disease or condition.

In a further example, distinct reference profiles can represent the diagnosis of a given disease or condition as taught herein vs. the diagnosis no such disease or condition (such as, e.g., the diagnosis of healthy, recovered from said disease or condition, etc.). In another example, distinct reference profiles may represent the diagnosis of said disease or condition of varying degree or severity.

In a yet another example, distinct reference profiles may represent a good prognosis for a disease or condition as taught herein vs. a poor prognosis for said disease or condition. In a further example, distinct reference profiles may represent varyingly favourable or unfavourable prognoses for such disease or condition.

Reference profiles used herein may be established according to known procedures previously employed for other biomarkers.

For example, a reference profile of the quantity and/or activity of procathepsin L, cathepsin L or a fragment thereof and the presence or absence and/or quantity of one or more other biomarkers such as lactate for a particular prediction, diagnosis and/or prognosis of a given disease or condition as taught herein may be established by determining the profile in sample(s) from one individual or from a population of individuals characterised by said particular prediction, diagnosis and/or prognosis of said disease or condition (i.e., for whom said prediction, diagnosis and/or prognosis of said disease or condition holds true). Such population may comprise without limitation 2, 10, 100, or even several hundreds or more individuals.

Hence, by means of an illustrative example, reference profiles for the diagnoses of a given disease or condition as taught herein vs. no such disease or condition may be established by determining the biomarker profiles in sample(s) from one individual or from a population of individuals diagnosed as, respectively, having or not having said disease or condition.

In an embodiment the present methods may include a step of establishing such reference profile(s). In an embodiment, the present kits and devices may include means for establishing a reference profile for a particular prediction, diagnosis and/or prognosis of a given disease or condition as taught herein. Such means may for example comprise one or more samples (e.g., separate or pooled samples) from one or more individuals characterised by said particular prediction, diagnosis and/or prognosis of said disease or condition.

Further, art-known multi-parameter analyses may be employed mutatis mutandis to determine deviations between groups of values and profiles generated there from (e.g., between sample and reference biomarker profiles).

When a deviation is found between the sample profile and a reference profile representing a certain prediction, diagnosis and/or prognosis of a given disease or condition as taught herein, said deviation is indicative of or may be attributed to the conclusion that the prediction, diagnosis and/or prognosis of said disease or condition in said subject is different from that represented by the reference profile.

When no deviation is found between the sample profile and a reference profile representing a certain prediction, diagnosis and/or prognosis of a given disease or condition as taught herein, the absence of such deviation is indicative of or may be attributed to the conclusion that the prediction, diagnosis and/or prognosis of said disease or condition in said subject is substantially the same as that represented by the reference profile.

The present invention further provides kits or devices for the diagnosis, prediction, prognosis and/or monitoring of any one disease or condition as taught herein comprising means for detecting the level of any one or more biomarkers in a sample of a subject. In a more preferred embodiment, such a kit or kits of the invention can be used in clinical settings or at home. The kits as taught herein may be used for diagnosing said disease or condition, for monitoring the effectiveness of treatment of a subject suffering from said disease or condition with an agent, or for preventive monitoring or screening of subjects for the occurrence of said disease or condition in said subject. For instance, the kits as taught herein may be used for assessing or monitoring the risk of developing ischemia-related complications in a subject having one or more the conditions selected from atherosclerosis, diabetes, obesity, ischemic heart disease, chronic heart failure, heart valve problems, lipid disorders, lipoprotein disorders, and claudicatio.

In a clinical setting, the kit or device may be in the form of a bed-side device or in an ICU or ED team setting, e.g. as part of the equipment of an ambulance or other moving emergency vehicle or team equipment or as part of a first-aid kit. The diagnostic kit or device may assist a medical practitioner, a first aid helper, or nurse to decide whether the patient under observation is developing a disease or condition as taught herein, after which appropriate action or treatment can be started immediately.

A home-test kit gives the patient a readout which he can communicate to a medicinal practitioner, a first aid helper or to the emergency department of a hospital, after which appropriate action can be taken. Such a home-test device is of particular interest for people having either a history of, or are at risk of suffering from any one disease or condition as taught herein.

Typical kits or devices according to the invention comprise the following elements:

a) a means for obtaining a sample from the subject and
b) a means or device for measuring the amount of any one or more markers as taught herein in said sample and visualizing whether the amount of the one or more markers in said sample is below or above a certain threshold level or value, indicating whether the subject is suffering from a given disease or condition as taught herein or not.

In any of the embodiments of the invention, the kits or devices may additionally comprise c) means for communicating directly with a medical practitioner, an emergency department of the hospital or a first aid post, indicating that a person is suffering from said disease or condition or not.

The term “threshold level or value” or “reference value” is used interchangeably as a synonym and is as defined herein. It may also be a range of base-line (e.g. “dry weight”) values determined in an individual patient or in a group of patients with highly similar disease conditions, taken at about the same time of gestation.

Without wanting to be bound by any theory, the inventors saw that the level of procathepsin L, cathepsin L or a fragment thereof is increased in case of ischemia. As shown in the example section, the protein level of procathepsin L was 2.4 times higher 24 hours post-surgery in patients who died 90 days after surgery compared with subjects who survived 90 days after surgery.

The threshold value indicated in the present invention is therefore more to be seen as a value in a reference.

Any of kits as defined herein may be used as a bed-side device for use by the subject himself or by a clinical practitioner.

Non-limiting examples are: systems comprising specific binding molecules for said one or more markers attached to a solid phase, e.g. lateral flow strips or dipstick devices and the like well known in the art. One non-limiting example to perform a biochemical assay is to use a test-strip and labelled antibodies which combination does not require any washing of the membrane. The test strip is well known, for example, in the field of pregnancy testing kits where an anti-hCG antibody is present on the support, and is carried complexed with hCG by the flow of urine onto an immobilised second antibody that permits visualisation. Other non-limiting examples of such home test devices, systems or kits can be found for example in the following U.S. Pat. Nos. 6,107,045, 6,974,706, 5,108,889, 6,027,944, 6,482,156, 6,511,814, 5,824,268, 5,726,010, 6,001,658 or U.S. patent applications: 2008/0090305 or 2003/0109067. In a preferred embodiment, the invention provides a lateral flow device or dipstick. Such dipstick comprises a test strip allowing migration of a sample by capillary flow from one end of the strip where the sample is applied to the other end of such strip where presence of an analyte in said sample is measured. In another embodiment, the invention provides a device comprising a reagent strip. Such reagent strip comprises one or more test pads which when wetted with the sample, provide a colour change in the presence of an analyte and/or indicate the concentration of the protein in said sample.

In order to obtain a semi-quantitative test strip in which only a signal is formed once the level of any one or more markers in the sample is higher than a certain predetermined threshold level or value, a predetermined amount of fixed capture antibodies for procathepsin L, cathepsin L or a fragment thereof can be present on the test strip. This enables the capture of a certain amount of procathepsin L, cathepsin L or a fragment thereof present in the sample, corresponding to the threshold level or value as predetermined. The remaining amount of procathepsin L, cathepsin L or a fragment thereof (if any) bound by e.g. a conjugated or labelled binding molecules can then be allowed to migrate to a detection zone which subsequently only produces a signal if the level of said one or more biomarkers in the sample is higher than the predetermined threshold level or value.

Another possibility to determine whether the amount of any one or more markers in the sample is below or above a certain threshold level or value, is to use a primary capturing antibody capturing all of said one or more markers protein present in the sample, in combination with a labelled secondary antibody, developing a certain signal or colour when bound to the solid phase. The intensity of the colour or signal can then either be compared to a reference colour or signal chart indicating that when the intensity of the signal is above a certain threshold signal, the test is positive. Alternatively, the amount or intensity of the colour or signal can be measured with an electronic device comprising e.g. a light absorbance sensor or light emission meter, resulting in a numerical value of signal intensity or colour absorbance formed, which can then be displayed to the subject in the form of a negative result if said numerical value is below the threshold value or a positive result if said numerical value is above the threshold value. This embodiment is of particular relevance in monitoring the level of said one or more markers in a patient over a period of time.

The reference value or range can e.g. be determined using the home device in a period wherein the subject is free of a given disease or condition, giving the patient an indication of his base-line level of any one or more markers. Regularly using the home test device will thus enable the subject to notice a sudden change in levels of said one or more markers as compared to the base-line level, which can enable him to contact a medical practitioner.

Alternatively, the reference value can be determined in the subject suffering from a given disease or condition as taught herein, which then indicates his personal “risk level” for any one or more markers, i.e. the level of said one or more markers which indicates he is or will soon be exposed to said disease or condition. This risk level is interesting for monitoring the disease progression or for evaluating the effect of the treatment.

Furthermore, the reference value or level can be established through combined measurement results in subjects with highly similar disease states or phenotypes (e.g. all having no disease or condition as taught herein or having said disease or condition).

Non-limiting examples of semi-quantitative tests known in the art, the principle of which could be used for the home test device according to the present invention are the HIV/AIDS test or Prostate Cancer tests sold by Sanitoets. The home prostate test is a rapid test intended as an initial semi-quantitative test to detect PSA blood levels higher than 4 ng/ml in whole blood. The typical home self-test kit comprises the following components: a test device to which the blood sample is to be administered and which results in a signal when the protein level is above a certain threshold level, an amount of diluent e.g. in dropper pipette to help the transfer of the analytes (i.e. the protein of interest) from the sample application zone to the signal detection zone, optionally an empty pipette for blood specimen collection, a finger pricking device, optionally a sterile swab to clean the area of pricking and instructions of use of the kit.

Similar tests are also known for e.g. breast cancer detection and CRP-protein level detection in view of cardiac risk home tests. The latter test encompasses the sending of the test result to a laboratory, where the result is interpreted by a technical or medical expert. Such telephone or internet based diagnosis of the patient's condition is of course possible and advisable with most of the kits, since interpretation of the test result is often more important than conducting the test. When using an electronic device as mentioned above which gives a numerical value of the level of protein present in the sample, this value can of course easily be communicated through telephone, mobile telephone, satellite phone, E-mail, internet or other communication means, warning a hospital, a medicinal practitioner or a first aid team that a person is, or may be at risk of, suffering from the disease or condition as taught herein. A non-limiting example of such a system is disclosed in U.S. Pat. No. 6,482,156.

The presence and/or concentration of procathepsin L, cathepsin L or a fragment thereof in a sample can be measured by surface plasmon resonance (SPR) using a chip having binding molecule for procathepsin L, cathepsin L or a fragment thereof immobilized thereon, fluorescence resonance energy transfer (FRET), bioluminescence resonance energy transfer (BRET), fluorescence quenching, fluorescence polarization measurement or other means known in the art. Any of the binding assays described can be used to determine the presence and/or concentration of procathepsin L, cathepsin L or a fragment thereof in a sample. To do so, binding molecule for procathepsin L, cathepsin L or a fragment thereof is reacted with a sample, and the concentration of procathepsin L, cathepsin L or a fragment thereof is measured as appropriate for the binding assay being used. To validate and calibrate an assay, control reactions using different concentrations of standard procathepsin L, cathepsin L or a fragment thereof and/or binding molecule for procathepsin L, cathepsin L or a fragment thereof can be performed. Where solid phase assays are employed, after incubation, a washing step is performed to remove unbound procathepsin L, cathepsin L or a fragment thereof. Bound, procathepsin L, cathepsin L or a fragment thereof is measured as appropriate for the given label (e.g., scintillation counting, fluorescence, antibody-dye etc.). If a qualitative result is desired, controls and different concentrations may not be necessary. Of course, the roles of procathepsin L, cathepsin L or a fragment thereof and binding molecule for procathepsin L, cathepsin L or a fragment thereof may be switched; the skilled person may adapt the method so binding molecule for procathepsin L, cathepsin L or a fragment thereof is applied to sample, at various concentrations of sample.

A “binding molecule for procathepsin L, cathepsin L or a fragment thereof” according to the invention is any substance that binds specifically to procathepsin L, cathepsin L or a fragment thereof. Examples of a binding molecule for procathepsin L, cathepsin L or a fragment thereof useful according to the present invention, includes, but is not limited to an antibody, a polypeptide, a peptide, a lipid, a carbohydrate, a nucleic acid, peptide-nucleic acid, small molecule, small organic molecule, or other drug candidate. A binding molecule for procathepsin L, cathepsin L or a fragment thereof can be natural or synthetic compound, including, for example, synthetic small molecule, compound contained in extracts of animal, plant, bacterial or fungal cells, as well as conditioned medium from such cells. Alternatively, binding molecule for procathepsin L, cathepsin L or a fragment thereof can be an engineered protein having binding sites for procathepsin L, cathepsin L or a fragment thereof. According to an aspect of the invention, a binding molecule for procathepsin L, cathepsin L or a fragment thereof binds specifically to procathepsin L, cathepsin L or a fragment thereof with an affinity better than 10⁻⁶ M. A suitable binding molecule for procathepsin L, cathepsin L or a fragment thereof can be determined from its binding with a standard sample of procathepsin L, cathepsin L or a fragment thereof. Methods for determining the binding between binding molecule for procathepsin L, cathepsin L or a fragment thereof and procathepsin L, cathepsin L or a fragment thereof are known in the art. As used herein, the term antibody includes, but is not limited to, polyclonal antibodies, monoclonal antibodies, humanised or chimeric antibodies, engineered antibodies, and biologically functional antibody fragments (e.g. scFv, nanobodies, Fv, etc) sufficient for binding of the antibody fragment to the protein. Such antibody may be commercially available antibody against procathepsin L, cathepsin L or a fragment thereof, such as, for example, a mouse, rat, human or humanised monoclonal antibody.

In a preferred embodiment, the binding molecule or agent is capable of binding both the mature membrane- or cell-bound protein or fragment of procathepsin L or cathepsin L. In a more preferred embodiment, the binding agent or molecule is specifically binding or detecting the soluble form, preferably the plasma circulating form of procathepsin L, cathepsin L or a fragment thereof, as defined herein.

According to one aspect of the invention, the binding molecule fro procathepsin L, cathepsin L or a fragment thereof is labelled with a tag that permits detection with another agent (e.g. with a probe binding partner). Such tags can be, for example, biotin, streptavidin, his-tag, myc tag, maltose, maltose binding protein or any other kind of tag known in the art that has a binding partner. Example of associations which can be utilised in the probe:binding partner arrangement may be any, and includes, for example biotin:streptavidin, his-tag:metal ion (e.g. Ni²⁺), maltose:maltose binding protein.

The specific-binding agents, peptides, polypeptides, proteins, biomarkers etc. in the present kits may be in various forms, e.g., lyophilised, free in solution or immobilised on a solid phase. They may be, e.g., provided in a multi-well plate or as an array or microarray, or they may be packaged separately and/or individually. The may be suitably labelled as taught herein. Said kits may be particularly suitable for performing the assay methods of the invention, such as, e.g., immunoassays, ELISA assays, mass spectrometry assays, and the like.

The term “modulate” generally denotes a qualitative or quantitative alteration, change or variation specifically encompassing both increase (e.g., activation) or decrease (e.g., inhibition), of that which is being modulated. The term encompasses any extent of such modulation.

For example, where modulation effects a determinable or measurable variable, then modulation may encompass an increase in the value of said variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of said variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation.

Preferably, modulation of the activity and/or level of intended target(s) (e.g., procathepsin L gene or protein, or cathepsin L gene or protein) may be specific or selective, i.e., the activity and/or level of intended target(s) may be modulated without substantially altering the activity and/or level of random, unrelated (unintended, undesired) targets.

Reference to the “activity” of a target such as procathepsin L or cathepsin L protein may generally encompass any one or more aspects of the biological activity of the target, such as without limitation any one or more aspects of its biochemical activity, enzymatic activity, signalling activity and/or structural activity, e.g., within a cell, tissue, organ or an organism.

In the context of therapeutic or prophylactic targeting of a target, the reference to the “level” of a target such as procathepsin L or cathepsin L gene or protein may preferably encompass the quantity and/or the availability (e.g., availability for performing its biological activity) of the target, e.g., within a cell, tissue, organ or an organism.

For example, the level of a target may be modulated by modulating the target's expression and/or modulating the expressed target. Modulation of the target's expression may be achieved or observed, e.g., at the level of heterogeneous nuclear RNA (hnRNA), precursor mRNA (pre-mRNA), mRNA or cDNA encoding the target. By means of example and not limitation, decreasing the expression of a target may be achieved by methods known in the art, such as, e.g., by transfecting (e.g., by electroporation, lipofection, etc.) or transducing (e.g., using a viral vector) a cell, tissue, organ or organism with an antisense agent, such as, e.g., antisense DNA or RNA oligonucleotide, a construct encoding the antisense agent, or an RNA interference agent, such as siRNA or shRNA, or a ribozyme or vectors encoding such, etc. By means of example and not limitation, increasing the expression of a target may be achieved by methods known in the art, such as, e.g., by transfecting (e.g., by electroporation, lipofection, etc.) or transducing (e.g., using a viral vector) a cell, tissue, organ or organism with a recombinant nucleic acid which encodes said target under the control of regulatory sequences effecting suitable expression level in said cell, tissue, organ or organism. By means of example and not limitation, the level of the target may be modulated via alteration of the formation of the target (such as, e.g., folding, or interactions leading to formation of a complex), and/or the stability (e.g., the propensity of complex constituents to associate to a complex or disassociate from a complex), degradation or cellular localisation, etc. of the target.

In a preferred embodiment, said modulation leads to a decrease in activity of procathepsin L or cathepsin L, either by inactivating or blocking its function at the protein level or by preventing transcription and translation of the coding sequence of procathepsin L or cathepsin L into its protein, i.e. at the mRNA or gene level. Since it is clear that the level of procathepsin L or cathepsin L is increased in subjects suffering form ischemia as defined herein, decreasing the activity of procathepsin L or cathepsin L intends to normalise and/or improve the condition of the subject.

The term “antisense” generally refers to a molecule designed to interfere with gene expression and capable of specifically binding to an intended target nucleic acid sequence. Antisense agents typically encompass an oligonucleotide or oligonucleotide analogue capable of specifically hybridising to the target sequence, and may typically comprise, consist essentially of or consist of a nucleic acid sequence that is complementary or substantially complementary to a sequence within genomic DNA, hnRNA, mRNA or cDNA, preferably mRNA or cDNA corresponding to the target nucleic acid. Antisense agents suitable herein may typically be capable of hybridising to their respective target at high stringency conditions, and may hybridise specifically to the target under physiological conditions.

The term “ribozyme” generally refers to a nucleic acid molecule, preferably an oligonucleotide or oligonucleotide analogue, capable of catalytically cleaving a polynucleotide. Preferably, a “ribozyme” may be capable of cleaving mRNA of a given target protein, thereby reducing translation thereof. Exemplary ribozymes contemplated herein include, without limitation, hammer head type ribozymes, ribozymes of the hairpin type, delta type ribozymes, etc. For teaching on ribozymes and design thereof, see, e.g., U.S. Pat. No. 5,354,855, U.S. Pat. No. 5,591,610, Pierce et al. 1998 (Nucleic Acids Res 26: 5093-5101), Lieber et al. 1995 (Mol Cell Biol 15: 540-551), and Benseler et al. 1993 (J Am Chem Soc 115: 8483-8484).

“RNA interference” or “RNAi” technology is routine in the art, and suitable RNAi agents intended herein may include inter alia short interfering nucleic acids (siNA), short interfering RNA (sRNA), double-stranded RNA (dsRNA), micro-RNA (miRNA), and short hairpin RNA (shRNA) molecules as known in the art. For teaching on RNAi molecules and design thereof, see inter alia Elbashir et al. 2001 (Nature 411: 494-501), Reynolds et al. 2004 (Nat Biotechnol 22: 326-30), http://rnaidesigner.invitrogen.com/rnaiexpress, Wang & Mu 2004 (Bioinformatics 20: 1818-20), Yuan et al. 2004 (Nucleic Acids Res 32(Web Server issue): W130-4), by M Sohail 2004 (“Gene Silencing by RNA Interference: Technology and Application”, 1^{st} ed., CRC, ISBN 0849321417), U Schepers 2005 (“RNA Interference in Practice: Principles, Basics, and Methods for Gene Silencing in C. elegans, Drosophila, and Mammals”, 1^{st} ed., Wiley-VCH, ISBN 3527310207), and D R Engelke & J J Rossi 2005 (“Methods in Enzymology, Volume 392: RNA Interference”, 1^{st} ed., Academic Press, ISBN 0121827976).

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

The present active substances (agents) may be used alone or in combination with any therapies known in the art for the disease and conditions as taught herein (“combination therapy”). Combination therapies as contemplated herein may comprise the administration of at least one active substance of the present invention and at least one other pharmaceutically or biologically active ingredient. Said present active substance(s) and said pharmaceutically or biologically active ingredient(s) may be administered in either the same or different pharmaceutical formulation(s), simultaneously or sequentially in any order.

The dosage or amount of the present active substances (agents) used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 pg/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every two or three weeks.

As used herein, a phrase such as “a subject in need of treatment” includes subjects that would benefit from treatment of a given disease or condition as taught herein. Such subjects may include, without limitation, those that have been diagnosed with said condition, those prone to contract or develop said condition and/or those in whom said condition is to be prevented.

The terms “treat” or “treatment” encompass both the therapeutic treatment of an already developed disease or condition, as well as prophylactic or preventative measures, wherein the aim is to prevent or lessen the chances of incidence of an undesired affliction, such as to prevent the chances of contraction and progression of a disease or condition as taught herein. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician. The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the present compounds.

The above aspects and embodiments are further supported by the following non-limiting examples.

### EXAMPLES

### Example 1

### MASSterclass Targeted Protein Quantitation for Early Validation of Candidate Markers Derived from Discovery

### MASSTERCLASS Experimental Setup

MASSterclass assays use targeted tandem mass spectrometry with stable isotope dilution as an end-stage peptide quantitation system (also called Multiple Reaction Monitoring (MRM) and Single Reaction Monitoring (SRM). The targeted peptide is specific (i.e., proteotypic) for the specific protein of interest. i.e., the amount of peptide measured is directly related to the amount of protein in the original sample. To reach the specificity and sensitivity needed for biomarker quantitation in complex samples, peptide fractionations precede the end-stage quantitation step.

A suitable MASSTERCLASS assay may include the following steps:
Plasma or serum sample
Depletion of human albumin and IgG (complexity reduction on protein level) using affinity capture with anti-albumin and anti-IgG antibodies using ProteoPrep spin columns (Sigma Aldrich)
Spiking of known amounts of isotopically labelled peptides. This peptide has the same amino acid sequence as the proteotypic peptide of interest, typically with one isotopically labelled amino acid built in to generate a mass difference. During the entire process, the labelled peptide has identical chemical and chromatographic behaviour as the endogenous peptide, except during the end-stage quantitation step which is based on molecular mass.
Tryptic digest. The proteins in the depleted serum or plasma sample are digested into peptides using trypsin. This enzyme cleaves proteins C-terminally from lysine and arginine, except when a proline is present C-terminally of the lysine or arginine. Before digestion, proteins are denatured by boiling, which renders the protein molecule more accessible for the trypsin activity during the 16 h incubation at 37° C.
First peptide-based fractionation: Free Flow Electrophoresis (FFE; BD Diagnostic) is a gel-free, fluid separation technique in which charged molecules moving in a continuous laminar flow are separated through an electrical field perpendicular to the flow. The electrical field causes the charged molecules to separate in the pH gradient according to their isoelectric point (pI). Only those fractions containing the monitored peptides are selected for further fractionation and LC-MS/MS analysis. Each peptide of interest elutes from the FFE chamber at a specific fraction number, which is determined during protein assay development using the synthetic peptide homologue. Specific fractions or fraction pools (multiplexing) proceed to the next level of fractionation.
Second peptide-based fractionation: Phenyl HPLC(XBridge Phenyl; Waters) separates peptides according to hydrophobicity and aromatic nature of amino acids present in the peptide sequence. Orthogonality with the back-end C18 separation is achieved by operating the column at an increased pH value (pH 10). As demonstrated by Gilar et al., 2005 (J. Sep. Sci., 28(14): 1694-1703), pH is by far the most drastic parameter to alter peptide selectivity in RP-HPLC. Each peptide of interest elutes from the Phenyl column at a specific retention time, which is determined during protein assay development using the synthetic peptide homologue. The use of an external control system, in which a mixture of 9 standard peptides is separated upfront a batch of sample separations, allows adjusting the fraction collection in order to correct for retention time shifts. The extent of fractionation is dependent on the concentration of the protein in the sample and the complexity of that sample.
LC-MS/MS based quantification, including further separation on reversed phase (C18) nanoLC (PepMap C18; Dionex) and MS/MS: tandem mass spectrometry using MRM (4000 QTRAP; ABI)/SRM (Vantage TSQ; Thermo Scientific) mode. The LC column is connected to an electrospray needle connected to the source head of the mass spectrometer. As material elutes from the column, molecules are ionized and enter the mass spectrometer in the gas phase. The peptide that is monitored is specifically selected to pass the first quadrupole (Q1), based on its mass to charge ratio (m/z). The selected peptide is then fragmented in a second quadrupole (Q2) which is used as a collision cell.

The resulting fragments then enter the third quadrupole (Q3). Depending on the instrument settings (determined during the assay development phase) only a specific peptide fragment or specific peptide fragments (or so called transitions) are selected for detection.
The combination of the m/z of the monitored peptide and the m/z of the monitored fragment of this peptide is called a transition. This process can be performed for multiple transitions during one experiment. Both the endogenous peptide (analyte) and its corresponding isotopically labelled synthetic peptide (internal standard) elute at the same retention time, and are measured in the same LC-MS/MS experiment.
The MASSterclass readout is defined by the ratio between the area under the peak specific for the analyte and the area under the peak specific for the synthetic isotopically labelled analogue (internal standard). MASSterclass readouts are directly related to the original concentration of the protein in the sample. MASSterclass readouts can therefore be compared between different samples and groups of samples.

A typical MASSTERCLASS protocol followed in the present study is given here below:
25 μl of plasma is subjected to a depletion of human albumin and IgG (ProteoPrep spin columns; Sigma Aldrich) according to the manufacturer's protocol, except that 20 mM NH₄HCO₃ was used as the binding/equilibration buffer,
the depleted sample (225 μl) is denatured for 15 min at 95° C. and immediately cooled on ice,
500 fmol of the isotopically labelled peptide (custom made ‘Heavy AQUA’ peptide; Thermo Scientific) is spiked in the sample,
20 μg trypsin is added to the sample and digestion is allowed for 16 h at 37° C.,
The digested sample was first diluted 1/8 in solvent A (0.1% formic acid) and then 1/20 in the same solvent containing 250 amol/μl of all isotopically labelled peptides (custom made ‘Heavy AQUA’ peptide; Thermo Scientific) of interest,
20 μl of the final dilution was separated using reverse-phase NanoLC with on-line MS/MS in MRM/SRM mode:
   Column: PepMap C18, 75 μm I.D.×25 cm L, 100 Å pore diameter, 5 μm particle size
   Solvent A: 0.1% formic acid
   Solvent B: 80% acetonitrile, 0.1% formic acid
   Gradient: 30 min; 2%-55% Solvent B
   MS/MS in MRM mode: method contains the transitions for the analyte as well as for the synthetic, labelled peptide.
   The used transitions were experimentally determined and selected during protein assay development
   Each of the transitions of interest was measured for a period starting 3 minutes before and ending 3 minutes after the determined retention time of the peptide of interest, making sure that each peak had at least 15 datapoints.
The raw data was analysed and quantified using the LCQuan software (Thermo Scientific): the area under the analyte (=the procathepsin L peptide) peak and under the internal standard (the labelled, synthetic procathepsin L peptide) peak at the same C18 retention time was determined by automatic peak detection. These were cross-checked manually.
The MASSterclass readout was defined by the ratio of the analyte peak area and the internal standard peak area

TABLE 1
Peptides used for the different
MASSterclass assays

| | Peptide | Sequence |
|---|---|---|
| Marker/protein | sequence | ID |
| Procathepsin L | LYGMNEEGWR | 4 |
| Cystatin C | ALDFAVGEYNK | 5 |
| C-reactive protein (CRP) | ESDTSYVSLK | 6 |

### MASSTERCLASS Output

The measured ratios are differential quantities of peptides. In other words, the ratio is the normalised concentration of a peptide. The concentration of a peptide is proportional to the ratio measured in the mass spectrometer.

### Example 2

### Procathepsin L as a Biomarker for the Prediction of Mortality

This example demonstrates the clinical utility of procathepsin L measurement for the prediction of mortality in critically ill patients. The cohort used in the present study was a single centre cohort of cardiac surgery patients (n=100). Patients were selected to enrich for post-surgical incidence of acute kidney injury (AKI), i.e. patients were either elderly (more than 70 years of age), had pre-existing reduced kidney function or had compromised heart function. All patients either underwent coronary artery bypass graft surgery (CABG) or a valve repair or replacement. Post-surgical outcome for the patients was recorded as either mortality or incidence of AKI as defined by AKIN or RIFLE criteria as taught herein. Markers were measured pre-surgery (at induction) and 24 hrs post surgery using MASSterclass for procathepsin L, C reactive protein (CRP) and cystatin C, using clinical immuno-assay for NGAL (Bioporto Diagnostics, Gentofte, Denmark) or the standard enzymatic measurement method for serum lactate. For each marker, receiver operator characteristic (ROC) analysis was performed to calculate the performance to detect, predict or diagnose the different patient outcomes. The estimated and 95% confidence intervals for area under the curve (AUC) were also computed using the Delong method.

Detailed patient characteristics can be found in Table 2.

TABLE 2
Overview of patient characteristics

| Variables | Cardiac surgery patients (n = 100) |
|---|---|
| Age (average-yrs) | 70 |
| Gender (% male) | 75% |
| Type of surgery | |
| CABG | 52% |
| Valve repair/replacement | 76% |
| concomitant | 29% |
| Medical history | |
| NYHA III or IV | 26% |
| LVEF < 35% | 21% |
| COPD | 19% |
| IDDM | 5% |
| Pre-op eGfr | 61.5 (50.2-76.7) |
| Outcome | |
| Mortality | 12% |
| AKI (AKIN) | 50% |
| AKI (RIFLE) | 21% |

At 24 h post-surgery, procathepsin L levels were found to be 2.4 fold higher in patients who died during follow-up compared to survivors. For mortality prediction, procathepsin L reaches an AUC of 0.88 (0.78-0.95), comparable to lactate 0.89 (0.81-0.96) and significantly better than NGAL, cystatin C and CRP (Table 3). Mortality post-surgery of CABG or valve replacement or repair is most often caused by heart failure or cardiogenic shock due to insufficient perfusion of the whole body.

TABLE 3
Marker performances for mortality prediction

| Marker | AUC (95% CI) |
|---|---|
| Ngal (plasma) | 0.71 (0.52-0.87) |
| CRP | 0.61 (0.42-0.81) |
| Lactate | 0.89 (0.81-0.96) |
| Cystatin C | 0.64 (0.44-0.81) |
| Procathepsin L | 0.88 (0.78-0.95) |

### Example 3

### Procathepsin L as a Biomarker for the Prediction of Significant Acute Kidney Injury (AKI)

In the same cohort of patients as used in Example 2, procathepsin L levels measured 24 h post surgery were scored as predictor for acute kidney injury (AKI). AKI frequently occurs in cardiac surgery patients because of the insufficient perfusion post surgery. Kidneys are sensitive to perfusion changes, leading to ischemia and renal failure.

AKI was diagnosed using different criteria such as AKIN criteria or RIFLE criteria, both as described herein. As illustrated in Table 4, standard measures for AKI such as creatinin and the derived estimated glomerular filtration rate (eGfr) are good predictors for AKI as defined by AKIN criteria, but do not perform so well in predicting the more significant AKI as defined by RIFLE-R (risk) or RIFLE-I (injury). Procathepsin L levels do perform better to predict the more significant AKI cases as defined by the AKI criteria and are in this respect better than the emerging AKI marker NGAL.

TABLE 4
Marker performance to predict AKI post surgery
(marker levels were measured 24 hrs post
surgery and AKI was defined using different criteria)

| Marker | AKIN | RIFLE-R | RIFLE-I |
|---|---|---|---|
| Creatinin (micromol/l) | 0.84 | 0.73 | 0.66 |
| eGFR (MDRD formula) | 0.82 | 0.77 | 0.70 |
| Lactate (mmol/l) | 0.67 | 0.79 | 0.80 |
| Cystatin C (MASSterclass) | 0.75 | 0.78 | 0.79 |
| Procathepsin L (MASSterclass) | 0.74 | 0.88 | 0.87 |
| Ngal (plasma, ng/mL) | 0.70 | 0.74 | 0.69 |
| CRP (MASSterclass) | 0.49 | 0.62 | 0.47 |

### Example 4

### Relation of Procathepsin L Levels to Measures of Perfusion, Hypoxia and Ischemia

In the cohort of cardiac surgery patients, as described in Example 2, associations of procathepsin L levels with clinical parameters were computed using univariate statistical tests. Spearman's ranked test was used to compute correlation coefficients and Wilcoxon rank sum test was used for assessing whether two independent samples of observation originate from the same population.

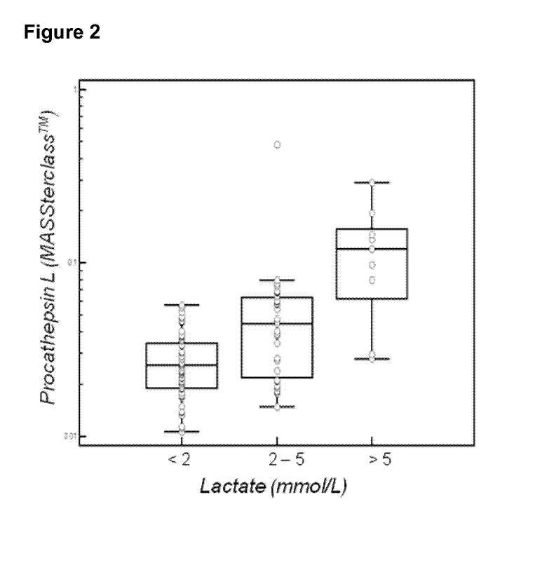

Procathepsin L levels showed a weak but significant correlation with serum lactate levels (r²=0.24; p=4×10⁻⁷). As illustrated in FIG. 2 procathepsin L levels show a nice stepwise increase going from normal lactate (<2 mmol/L) to increased lactate (2-5) to severely increased, indicative of major ischemia lactate levels (>5 mmol/L) Procathepsin L levels further show an inverse correlation with mean arterial pressure (r²=0.36; p=7.6×10⁻⁹). Furthermore, significantly higher procathepsin L levels were observed in patients treated with inotropes compared to patients not treated with inotropes. This analysis further corroborated the significant higher levels in patients who died during follow up and patients who developed AKI (as outlined in Examples 2 and 3). All these clinical measures mentioned are direct or indirect measures of (in)adequate perfusion.

### Example 5

### Procathepsin L and Lactate as a Powerful Biomarker for the Prediction of Ischemia-Related Complications

In the cohort of cardiac surgery patients, as described in Example 2, ischemia-related complications was defined as a patient who either died during follow-up or developed significant AKI (defined by RIFLE-R or worse). In total, 23 patients out of 100 had such an ischemia-related complications. Procathepsin L levels measured at 24 h post-surgery showed to be a good predictor of ischemia-related complications with an AUC of 0.91 (0.83-0.96), better than serum lactate with an AUC of 0.82 (0.73-0.89) (p=0.08) and CRP (p<0.0001), both reported predictors of ischemia-related complications in critical ill patients (Table 5).

TABLE 5
Summary of marker performances to predict
ischemia-related complications

| Marker | AUC (95% CI) |
|---|---|
| Lactate | 0.82 (0.73-0.89) |
| Procathepsin L | 0.91 (0.83-0.96) |
| Cystatin C | 0.76 (0.66-0.84) |
| CRP | 0.57 (0.47-0.67) |

Comparing procathepsin L levels measured pre- and post surgery, showed that procathepsin L levels were induced post-surgery in a discrete set of patients. In fact this set of patients corresponded with the patients with an ischemia-related complications (see FIG. 2). At a cut-off for maximum accuracy (corresponding to 3.3 fold increase), 75% of patients could be predicted with ischemia-related complications, with only 16% false positives.

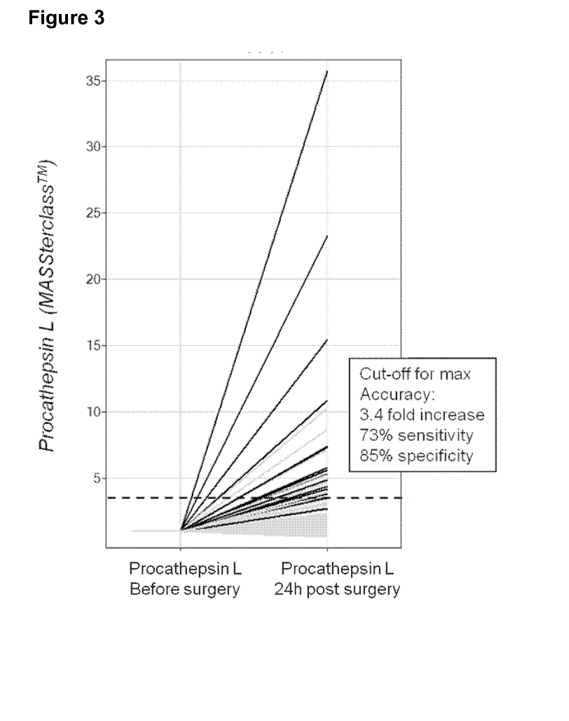

Comparing procathepsin L levels with lactate levels, showed that there was a correlation between the two markers, which was apparent in the high lactate levels above 4-5 mmol/l (see FIG. 3). In the lower ranges of lactate, there was no such correlation and thus measurement of procathepsin L levels can aid in the interpretation of lactate levels. Currently, lactate levels above 2 mmol/L are considered to be abnormal, but currently these levels are no trigger for therapy-based decisions because of the frequent false positive lactate elevations (for review: Jansen et al., Critical Care Medicine, 2009, 37 (10), 2827-2839). This was also observed in this cohort (see FIG. 2). Lactate at 2 mmol/l reached a sensitivity of 87% with a specificity of 78% for ischemia-related complications prediction. However, adding procathepsin L to lactate at 2 mmol/l increased the specificity to 94%, without significantly hampering the sensitivity. Hence, combined use of procathepsin L and lactate dramatically improved the accuracy to predict ischemia-related complications which advantageously aids the clinician in his therapeutic decision making.

### Example 6

### Procathepsin L Levels as a Goal for Treatment in Critically Ill Patients

In a cohort of critical ill patients wherein lactate is used as endpoint for goal-directed therapy or wherein lactate is used to guide therapy, procathepsin L levels are measured retrospectively. The relation between procathepsin L levels and mortality is established. Furthermore, the post-test probability of mortality after each procathepsin L measurement is compared to the post-test probability of each lactate measurement.

### Example 7

### (Pro)-Cathepsin L Levels as a Biomarker for the Prediction of Mortality and Significant Acute Kidney Injury (AKI)

In a cohort of critical ill patients, including critically ill patients with sepsis, SIRS, COPD, and patient after surgery, procathepsin L and cathepsin L levels are measured. The relation between procathepsin L or cathepsin L and mortality or AKI is established. For procathepsin L or cathepsin L, receiver operator characteristic (ROC) analysis is performed to calculate the performance to detect, predict or diagnose the different patient outcomes. The estimated and 95% confidence intervals for area under the curve (AUC) are also computed using the Delong method.

1. The in vitro use of procathepsin L or cathepsin L as a blood biomarker for diagnosing, predicting, prognosticating and/or monitoring ischemia in a subject.

2. The use according to claim 1, wherein said diagnosis, prediction, prognosis and/or monitoring ischemia comprises assessing the degree of ischemia in the subject.

3. The use according to claim 2, wherein the degree of ischemia in a subject is assessed in accordance with lactate levels.

4. The use according to claim 2 or 3, wherein the degree of ischemia is assessed as being:
(i) no ischemia,
(ii) low levels of ischemia with reversible or reparable physiological outcome which can lead to significant ischemic complications when left untreated, or
(iii) high levels of ischemia with potential irreversible or irreparable physiological damage, morbidity or mortality.

5. The use according to any one of claims 1 to 4, in combination with lactate as a biomarker.

6. An in vitro method for diagnosing, predicting, prognosticating and/or monitoring ischemia in a subject, wherein the examination phase of the method comprises measuring the quantity of procathepsin L or cathepsin L in a blood sample from the subject.

7. The method according to claim 6, wherein said diagnosis, prediction, prognosis and/or monitoring ischemia comprises assessing the degree of ischemia in the subject.

8. The method according to claim 7, wherein the degree of ischemia in a subject is assessed in accordance with lactate levels.

9. The method according to claim 7 or 8, wherein the degree of ischemia is assessed as being:
(i) no ischemia,
(ii) low levels of ischemia with reversible or reparable physiological outcome which can lead to significant ischemic complications when left untreated, or
(iii) high levels of ischemia with potential irreversible or irreparable physiological damage, morbidity or mortality.

10. The method according to any one of claims 6 to 9, wherein the examination phase of the method further comprises measuring the quantity of lactate in the blood sample from the subject.

11. The use or method according to any one of claims 1 to 10, wherein said diagnosis, prediction, prognosis and/or monitoring ischemia comprises distinguishing subjects with favourable outcome from subjects with ischemia-related complications.

12. The use or method according to any one of claims 1 to 11, wherein said ischemia-related complications are selected from the group consisting of: acute kidney injury (AKI), cardiogenic shock, myocardial infarction, heart failure, death, amputation or removal of the damaged area, organ or limb, brain infarction and its neurological deficits, and any organ damage or failure.

13. An in vitro method for diagnosing, predicting, prognosticating and/or monitoring acute kidney injury in a subject, wherein the examination phase of the method comprises measuring the quantity of procathepsin L or cathepsin L in a blood sample from the subject.

14. An in vitro method for predicting mortality in critically ill patients, wherein the examination phase of the method comprises measuring the quantity of procathepsin L or cathepsin L in a blood sample from the patient.

15. The method according to claim 14, wherein said critically ill patient is selected from the group consisting of patients presenting in intensive care units (ICU) or emergency departments (ED) with one or more of: serious trauma, systemic inflammatory response syndrome (SIRS), sepsis; severe sepsis, sepsis with organ dysfunction, septic shock, chronic obstructive pulmonary disease (COPD) with or without an acute exacerbation, patients having undergone surgery and more particularly cardiac surgery, complications from surgery, medical shock, bacterial, fungal or viral infections, Acute Respiratory Distress Syndrome (ARDS), pulmonary and systemic inflammation, pulmonary tissue injury, severe pneumonia, respiratory failure, acute respiratory failure, respiratory distress, subarachnoidal hemorrhage (SAH), (severe) stroke, asphyxia, neurological conditions, organ dysfunction, single or multi-organ failure (MOF), poisoning and intoxication, severe allergic reactions and anaphylaxis, burn injury, and acute cerebral hemorrhage or infarction.

16. The use or method according to any one of claims 1 to 15, wherein said subject has one or more of the conditions selected from atherosclerosis, diabetes, obesity, ischemic heart disease, chronic heart failure, heart valve problems, lipid disorders, lipoprotein disorders, and claudicatio.

17. The use or method according to any one of claims 1 to 16, wherein said monitoring ischemia comprises optimizing the training program of a subject.

18. The use or method according to any one of claims 1 to 17, for improving the interpretation of lactate levels in a sample of the subject.

19. The use or method according to any one of claims 1 to 18, for determining and/or steering the therapeutic intervention in the subject.

20. The use or method according to any one of claims 1 to 19, for assessing the impact of the therapeutic intervention.

21. The use or method according to any one of claims 1 to 20, for measuring the success of ischemic preconditioning in a subject who will undergo surgery or transplantation.

22. The use or method according to any one of claims 1 to 21, wherein said sample is blood, serum or plasma.

23. The use or method according to any one of claims 1 to 22 for predicting ischemia-related conditions and outcomes in pregnant women such as: placental insufficiency, placental thrombosis, placental infarction, abruption placentae, Intra Uterine Growth Retardation, Small for Gestational Age children, neurological or intellectual sequellae in the babie(s), spontaneous abortion, premature contractions, premature labor and delivery, fetal deformations, fetal infection, mors in utero, or low birth weight.

24. The use or method according to any one of claims 1 to 22 in combination with lactate for detecting liver cell insufficiency by differentiating between abnormal lactate production and abnormal lactate metabolism,
wherein abnormal lactate production is indicated when both levels of lactate and procathepsin L, cathepsin L or a fragment thereof are elevated, and
wherein abnormal lactate metabolism is indicated when lactate levels are elevated but the level of procathepsin L, cathepsin L or a fragment thereof is in the normal range.

25. The use or method according to any one of claims 1 to 22, wherein the quantity of procathepsin L, cathepsin L or a fragment thereof is measured using a binding agent capable of specifically binding to procathepsin L, cathepsin L or a fragment thereof respectively, or wherein the quantity of procathepsin L, cathepsin L or a fragment thereof is measured using an immunoassay technology, using a mass spectrometry analysis method, using a chromatography method, using RNA analysis tools such as northern blotting, or (quantitative)RT-PCR, or using a combination of said methods.

26. Use of a kit comprising means for measuring the quantity of procathepsin L in a sample from a subject, for performing the method according to any one of claims 6 to 25.

27. The use according to claim 26, wherein the kit further comprises a reference value of the quantity of procathepsin L or means for establishing said reference value, wherein said reference value represents a known diagnosis, prediction and/or prognosis of ischemia.

28. The use according to claim 26 or 27, wherein said kit additionally comprises means for measuring the quantity of lactate in a sample of the subject.
